(19) United States
(12) Patent Application Publication
(10) Pub. No.: US 2001/0056075 A1
(43) Pub. Date: Dec. 27, 2001

(54) Chimeric polypeptides of serum albumin and uses related thereto

(76) Inventors: Jeno Gyuris, Winchester, MA (US); Lou Lamphere, Newton, MA (US)

Correspondence Address: ROPES & GRAY, ONE INTERNATIONAL PLACE, BOSTON, MA, 02110-2624, US

(21) Appl. No.: 09/764,918

(22) Filed: Jan. 18, 2001

Related U.S. Application Data
(63) Continuation-in-part of application No. 09/619,285, filed on Jul. 19, 2000.
(60) Provisional application No. 60/144,534, filed on Jul. 19, 1999.

(51) Int. Cl. A61K 39/00; A61K 48/00; C07K 14/765

(52) U.S. Cl. 514/44; 424/185.1; 530/363

(57) ABSTRACT

The present invention relates to chimeric polypeptides in which a serum albumin protein has been altered to include one or more biologically active heterologous peptide sequences. The chimeric polypeptides may exhibit therapeutic activity related to the heterologous peptide sequences coupled with the improved serum half-lives derived from the serum albumin protein fragments. Heterologous peptide sequences maybe chosen to promote any biological effect, including angiogenesis inhibition, antitumor activity, and induction of apoptosis. The therapeutic effect may be achieved by direct administration of the chimeric polypeptide, or by transfecting cells with a vector including a nucleic acid encoding such a chimeric polypeptide.

This application is a continuation-in-part of U.S. application Ser. No. 09/619,285, filed Jul. 19, 2000, which is based on U.S. Provisional Application No. 60/144,534, filed Jul. 19, 1999, the specification of which is hereby incorporated by reference in its entirety.

### BACKGROUND OF THE INVENTION

Recent advances in recombinant DNA technology have made available a wide range of biologically active peptides. Although in some instances molecular remodeling, for instance by ligated gene fusion or by site directed mutagenesis, has endowed such proteins with properties compatible with optimal activity, it is generally the case that effective use of these products can only be achieved through delivery systems.

Polypeptide therapeutic agents, despite their promise in a number of disease treatments, are readily decomposed by gastric juices and by intestinal proteinases such as pepsin and trypsin. As a result, when these polypeptides are orally administered, they are barely absorbed and produce no effective pharmacological action. In order to obtain the desired biological activity, the polypeptides are at present usually dispensed in injectable dosage forms. However, the injectable route is inconvenient and painful to the patient, particularly when administration must occur on a regular and frequent basis. Consequently, efforts have focused recently on alternative methods for administration of such polypeptides.

Such agents usually exhibit a short half-life in the circulation, being rapidly excreted through the kidneys or taken up by the reticuloendothelial system (RES) and other tissues. To compensate for such premature drug loss, larger doses are required so that sufficient amounts of drug can concentrate in areas in need of treatment. However, this is not only costly; it can also lead to toxicity and an immune response to the foreign protein. Sustained-release formulations (Putney, S. D. et al. Nature Biotechnology 1998, 16, 153-157) generally reduce the necessary dosage, but still depend on injection or more objectionable forms of delivery. A therapeutic protein with a longer half-life in the body would maintain a more stable blood level in much the same way as a sustained-release formulation, but would not entail the difficulties of preparing a sustained-release formulation and would require an even lower dosage because it is destroyed less quickly. For instance, cytokines such as interferon (IFN-gamma) and interleukin-2 (IL-2) would be more effective, less toxic and could be used in smaller quantities, if their presence in the circulation could be extended.

### SUMMARY OF THE INVENTION

One aspect of the present invention provides a chimeric polypeptide comprising a biologically active heterologous peptide fragment inserted into a serum albumin protein or a homolog thereof. The heterologous peptide fragment may optionally replace a portion of the serum albumin protein sequence. A peptide fragment which replaces a portion of the serum albumin protein sequence need not be of the same length as the fragment it replaces. A chimeric polypeptide according to this aspect may include more than one heterologous peptide fragment which replaces a portion of the serum albumin protein sequence. The included fragments may be identical, may be distinct sequences from a protein unrelated to serum albumin protein, or may be distinct sequences of unrelated origin.

A chimeric polypeptide of this aspect, for example, may comprise the structure A-B-C, wherein A represents a first fragment of a serum albumin protein or homolog thereof, B represents a biologically active heterologous peptide sequence, and C represents another fragment of a serum albumin protein or a homolog thereof. Similarly, a chimeric polypeptide may comprise the structure A-B-C-D-E, wherein A, C, and E represent fragments of a serum albumin protein and B and D represent identical biologically active heterologous peptide sequences, two different biologically active sequences of a protein unrelated to serum albumin protein, or two different biologically active sequences of two different proteins unrelated to serum albumin protein. Analogously, a chimeric polypeptide may comprise the structure A-B-C-D-E-F-G, wherein A, C, E, and G represent fragments of a serum albumin protein and B, D, and F represent identical biologically active heterologous peptide sequences, at least two different biologically active sequences of a protein unrelated to serum albumin protein, or at least two different biologically active sequences of two different proteins unrelated to serum albumin protein. In certain embodiments, a peptide fragment of serum albumin or a heterologous peptide sequence includes at least 6 amino acids, at least 12 amino acids, or at least 18 amino acids.

A chimeric polypeptide may comprise the structure (A-B-C)ₙ, e.g., —HN-(A-B-C)ₙ-CO— or H₂N-(A-B-C)ₙ-CO₂H, wherein A, independently for each occurrence, represents a fragment of serum albumin (SA), B, independently for each occurrence, represents a biologically active heterologous peptide sequence, C, independently for each occurrence, represents a second biologically active heterologous peptide sequence or a fragment of serum albumin (SA), and n is an integer greater than 0. In certain embodiments, a peptide fragment of serum albumin or a heterologous peptide sequence includes at least 6 amino acids, at least 12 amino acids, or at least 18 amino acids.

Alternatively, such a chimeric polypeptide may comprise an N-terminal fragment of a serum albumin protein or a homolog thereof, a biologically active heterologous peptide sequence, and a C-terminal fragment of a serum albumin protein or a homolog thereof. The heterologous peptide sequence may be between about 3 and about 500 or between about 4 and about 400 residues in length, preferably between about 4 and about 200 residues, more preferably between about 4 and 100 residues, and most preferably between about 4 and about 20 residues.

In one embodiment, the chimeric polypeptide has a half-life in the blood no less than 10 days, preferably no less than about 14 days, and most preferably no less than 50% of the half-life of the native serum albumin protein or homolog thereof.

In another embodiment, the heterologous peptide sequence is capable of binding to a cell surface receptor protein. Examples of such a receptor protein include a G protein-coupled receptor, a tyrosine kinase receptor, a cytokine receptor, an MIRR receptor, and an orphan receptor.

In another embodiment, the chimeric polypeptide is capable of binding to an extracellular receptor or ion channel. The chimeric polypeptide may be an agonist or an antagonist of an extracellular receptor or ion channel. The chimeric polypeptide of this embodiment may, for example, induce apoptosis, modulate cell proliferation, or modulate differentiation of cell types.

The invention also comprises a nucleic acid sequence which encodes a chimeric polypeptide as described above.

The invention further comprises a delivery vector, such as a viral or retroviral vector comprising a nucleic acid sequence encoding the chimeric polypeptide. Suitable vectors may include, for example, an adenovirus, an adeno-associated virus, a herpes simplex virus, a human immunodeficiency viruses, or a vaccinia virus.

The invention also comprises a pharmaceutical composition comprising a chimeric polypeptide as described above, and methods for treating a disease in an organism by administering an effective dose of such a pharmaceutical composition to the organism. In a currently preferred embodiment, a chimeric polypeptide according to the invention comprises a fragment of an angiogenesis-inhibiting protein, such as angiostatin or endostatin, as the heterologous peptide sequence and is capable of inhibiting angiogenesis. For example, a peptide fragment that inhibits angiogenesis and which may be incorporated into a subject polypeptide is RGD (Arg-Gly-Asp), or a sequence which includes the sequence RGD (e.g., VRGDF). Analogous methods may be used to modulate conditions such as cell proliferation, cell differentiation, and cell death.

In a currently preferred embodiment, the present invention provides a method of treating a disease in an organism by introducing into cells of the organism genetic material encoding a chimeric polypeptide protein comprising serum albumin protein or segments thereof and one or more therapeutic proteins or polypeptides or fragments thereof, such that the introduced genetic material is expressed by the transfected cells of the organism. Analogous methods may be used to modulate conditions such as cell proliferation, cell differentiation, and cell death.

In another aspect, the present invention provides a method for treating a disease in an organism by introducing genetic material encoding a chimeric polypeptide comprising serum albumin protein or segments thereof and one or more therapeutic proteins or polypeptides or fragments thereof into target cells ex vivo under conditions sufficient to cause the genetic material to be incorporated into the cell, thereby causing the cell to express the genetic material encoding said proteins or polypeptides. The target cells are then introduced into the host organism such that the introduced genetic material encoding said proteins or polypeptides is expressed by the target cells in the organism. The target cells may be selected from the group consisting of blood cells, skeletal muscle cells, smooth muscle cells, stem cells, skin cells, liver cells, secretory gland cells, hematopoietic cells, and marrow cells.

Another aspect of the present invention provides transfected cells comprising target cells which have been exposed to a delivery vector comprising a nucleic acid encoding the chimeric protein or polypeptide of this invention. These cells are preferably selected from the group consisting of blood cells, skeletal muscle cells, smooth muscle cells, stem cells, skin cells, liver cells, secretory gland cells, hematopoietic cells, and marrow cells.

### BRIEF DESCRIPTION OF THE FIGURES

FIG. 1 shows the tertiary structure of human serum albumin (HSA).

FIG. 2 illustrates the transfection of cells with mouse serum albumin (MSA)-Myc fusion constructs and successful expression of the fusion protein, as well as binding of MSA and Myc antibodies to MSA-Myc fusion proteins depending on the location of the heterologous sequence in the MSA protein.

FIG. 3 depicts inhibition of FGF-induced proliferation of bovine capillary endothelial cells by RGD peptide and by MSA-myc-RGD fusion proteins.

FIGS. 4A-I highlight loops of serum albumen which may be replaced with display therapeutic polypeptide sequences as described below.

FIG. 5 illustrates amino acid sequences for the display of therapeutic polypeptide sequences in the Cys⁵³-Cys⁶² loop of mouse serum albumen.

FIG. 6 depicts the inhibitory effects of mouse serum albumen proteins as set forth in FIG. 5 on bovine capillary endothelial (BCE) cells stimulated by FGF.

FIG. 7 illustrates the inhibitory effects of mouse serum albumen proteins as set forth in FIG. 5 on human umbilical vein endothelial cells (HUVECs) stimulated by FGF.

FIG. 8 shows the induction of apoptosis induced by MSA-RGD fusion protein in NCI 1869 human non-small cell lung carcinoma cell line.

### DETAILED DESCRIPTION OF THE INVENTION

The systems and methods disclosed herein are directed towards increasing the lifetime of therapeutic polypeptides in the bloodstream by creating chimeric polypeptides containing segments of serum albumin (SA) and segments of biologically active heterologous peptide sequences. SA is the major protein constituent of the circulatory system, has a half-life in the blood of about three weeks (Rothschild, M. A. et al. Hepatology 1988, 8, 385-401), and is present in quantity (40 g/L in the serum). It is also known that the normal adult human liver produces approximately 15 grams of human serum albumin (HSA) per day, or about 200 mg per kilogram of body weight. Serum albumin has no immunological activity or enzymatic function, and is a natural carrier protein used to transport many natural and therapeutic molecules. Fusion proteins wherein a therapeutic polypeptide has been covalently linked to serum albumin have been shown to have serum half-lives many times longer than the half-life of the therapeutic peptide itself (Syed, S. et al. Blood 1997, 89, 3243-3252; Yeh, P. et al. Proc. Natl. Acad. Sci. USA 1992, 89, 1904-1908). In both cited publications, the half-life of the fusion protein was more than 140 times greater than that of the therapeutic polypeptide itself, and approached the half-life of unfused serum albumin. Furthermore, the amino-terminal portion of serum albumin has been found to favor particularly efficient translocation and export of the fusion proteins in eukaryotic cells (PCT publication WO 90/13653). Generally, this means that such proteins are more efficiently secreted by a cell manufacturing such proteins than are the free therapeutic polypeptides themselves.

From a drug delivery standpoint, chimeric polypeptides of serum albumin proteins offer substantial promise because serum albumins are found in tissues and secretions throughout the body. It is known, for example, that serum albumin is responsible for the transport of compounds across organ-circulatory interfaces into such organs as the liver, intestine, kidney, and brain. Chimeric proteins of serum albumin may thus manifest their biological activity anywhere in the body, crossing even the daunting blood-brain barrier.

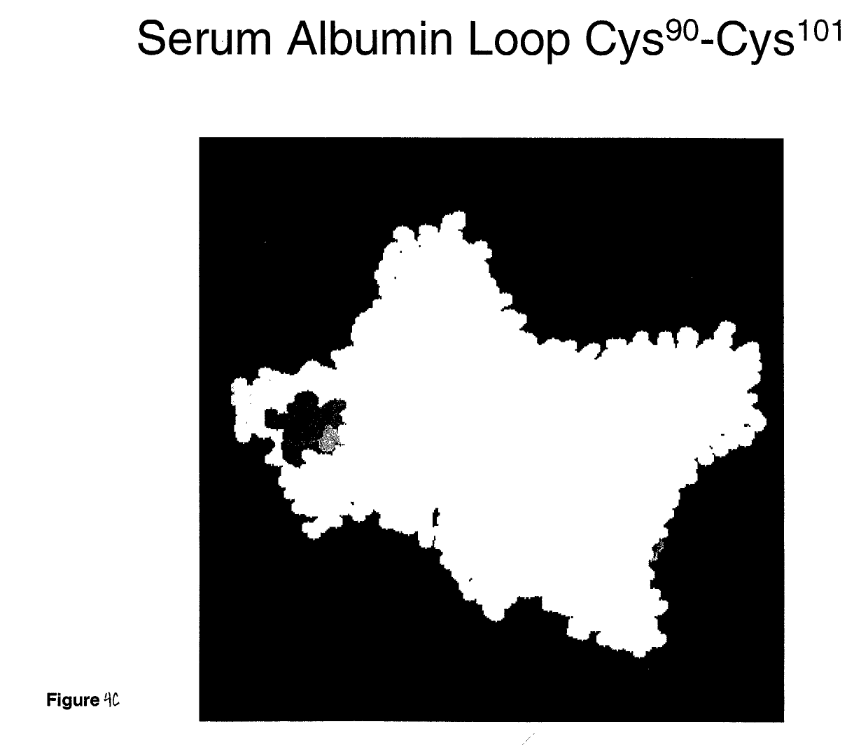

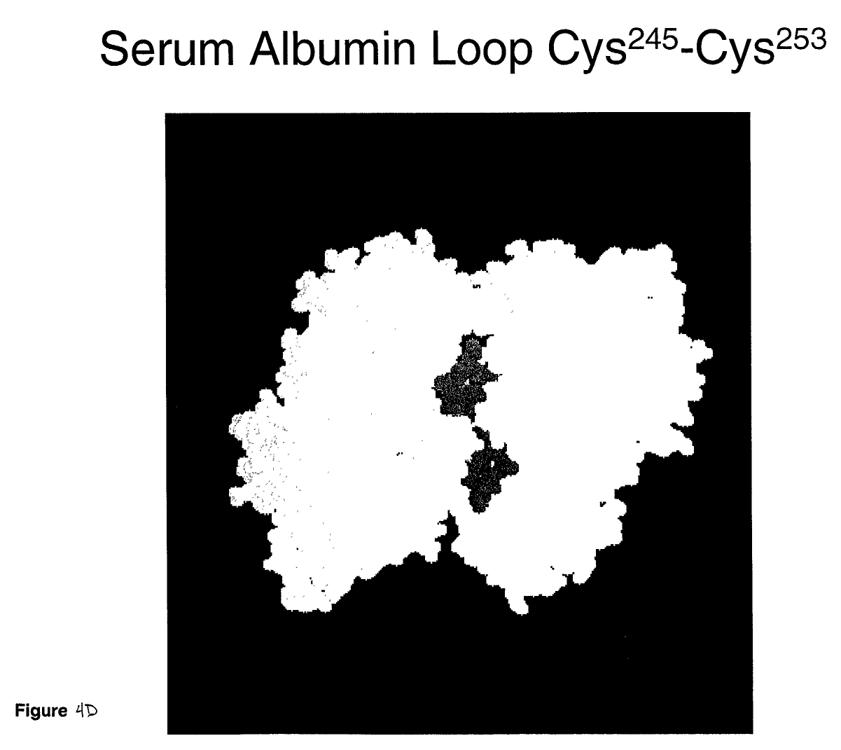

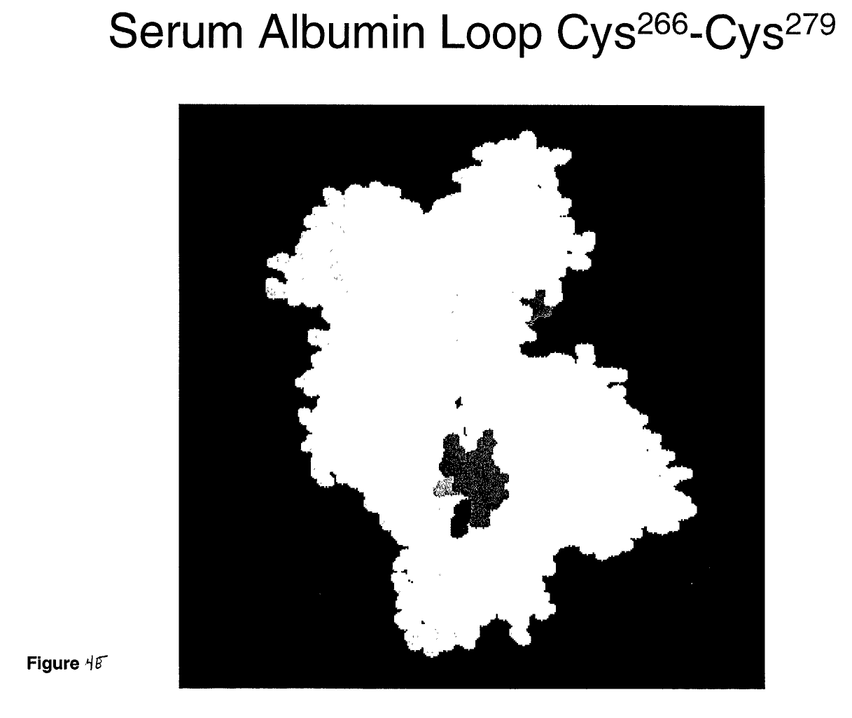

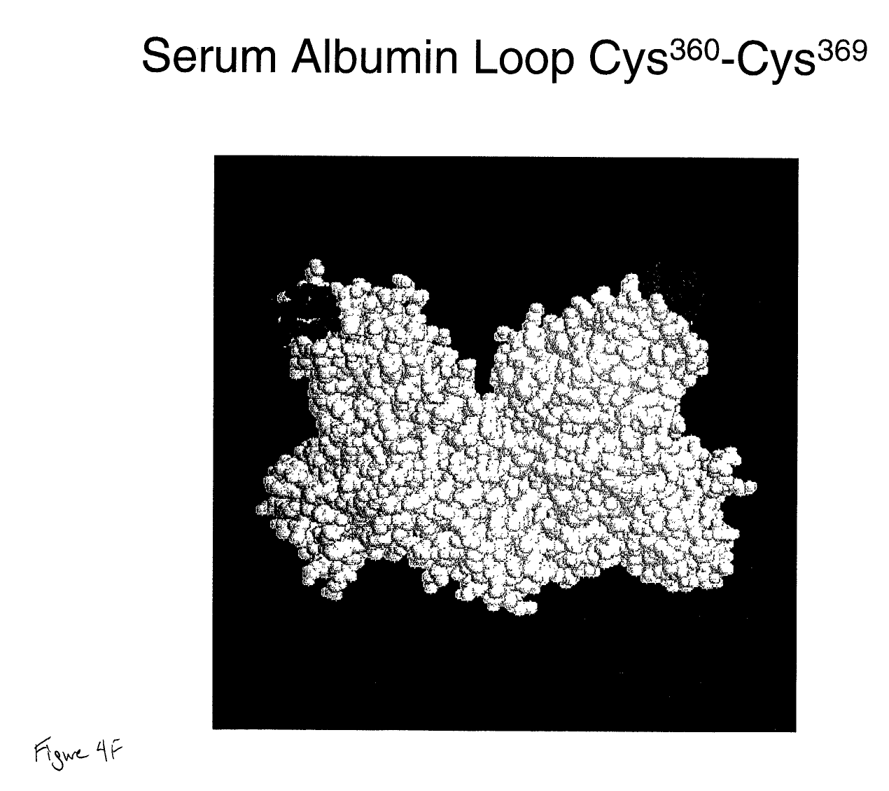

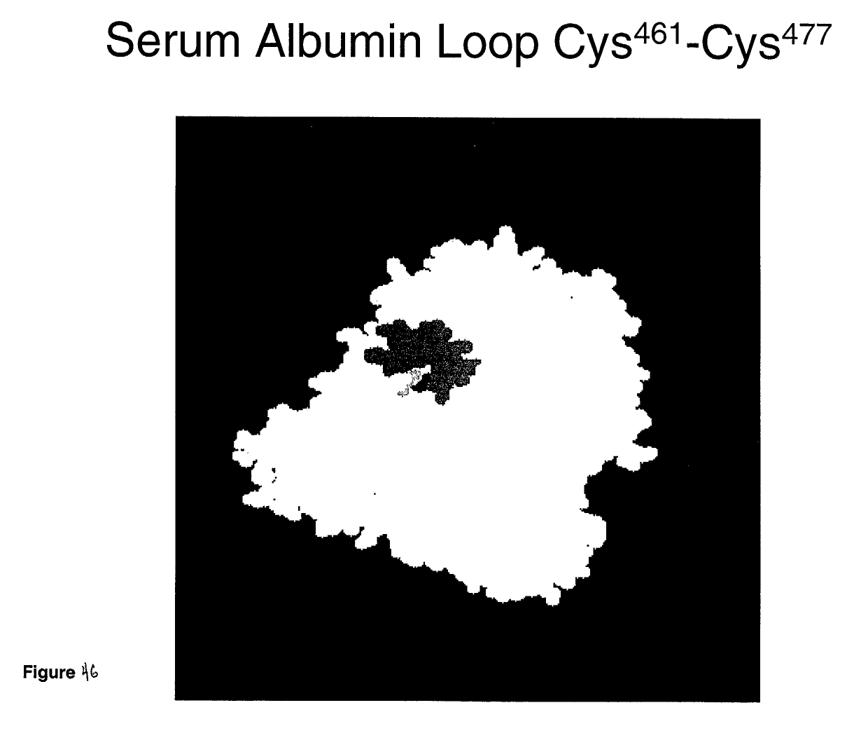

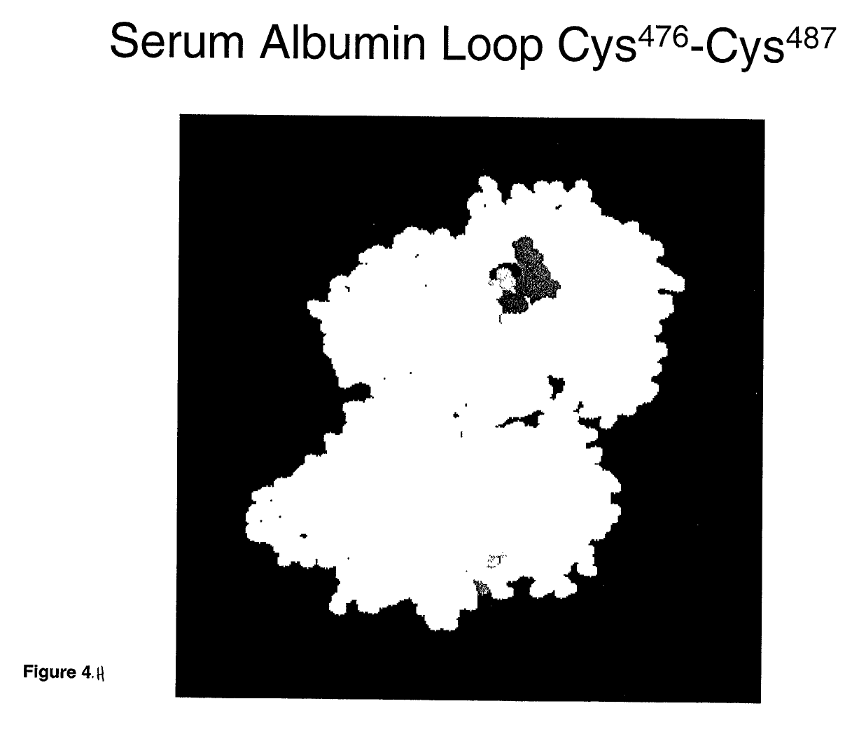

The three-dimensional structure and the chemistry of SA have been well studied (Carter, D. C. et al. Eur. J Biochem. 1994, 226, 1049-1052; He, X. M. et al. Nature 1992, 358, 209-215; Carter, D. C. et al. Science 1989, 244, 1195-1198). Thus, rather than relying on simple, binary fusion proteins as discussed above, portions of the SA protein may be strategically or combinatorially replaced by therapeutic polypeptides. For example, cysteine-constrained loops may be selected for replacement, e.g., on the presumption that structural changes to the loop are likely to minimally affect the tertiary structure of the protein as a whole. FIGS. 4A-I show the locations of several such loops on the mouse serum albumen protein. Effective replacement and insertion into such loops is demonstrated in the Examples below. The present invention contemplates insertion into or replacement of any one of the loops depicted in FIGS. 4A-I, or any combination of such loops. In certain embodiments, a loop selected for insertion or replacement is located at or near the surface of the serum albumen protein to facilitate intermolecular interactions. One of skill in the art will readily be able to adapt these techniques to other serum albumen proteins, e.g., bovine, human, and other serum albumen proteins.

Techniques of combinatorial mutagenesis combined with structurally motivated grafting procedures allow the random preparation of a library of many related polypeptides which carry a biologically active peptide fragment and are substantially similar to serum albumin in tertiary structure. For example, a chimeric polypeptide of the present invention may include a biologically active heterologous peptide sequence inserted into the peptide sequence of a serum albumin protein. The inserted sequence may optionally replace a portion of the serum albumin sequence, whether that portion is of similar or dissimilar length. In some cases, more than one insertion may be required to obtain the desired biological activity. Alternatively, a biologically active heterologous peptide sequence may be placed between two fragments of a serum albumin sequence to create such a chimeric polypeptide. Optionally, one or more additional biologically active peptide sequences may be placed between fragments of serum albumin protein. Chimeric polypeptides of the present invention may also be described as a biologically active heterologous peptide sequence flanked on one side by an N-terminal fragment of serum albumin protein and on the other side by a C-terminal fragment of serum albumin protein.

The advantage of such chimeric polypeptides is that the similarity to serum albumin protein in structure may camouflage these polypeptides to biological mechanisms which degrade foreign peptides even more effectively than known fusion proteins, because the foreign polypeptide fragments are carried on a protein that is substantially similar to a protein that is pervasive within the organism. Such proteins may retain the beneficial characteristics of serum albumin (non-immunogenicity, high level of expression, efficient secretion, and long half-life), while supporting the additional desired biological function.

Many therapeutic applications of such chimeric polypeptides will be obvious to those skilled in the art. For example, inclusion of a peptide fragment which inhibits cell proliferation might serve as a treatment for cancer and other diseases characterized by cell proliferation known to those in the art. Inclusion of a peptide fragment which modulates the differentiation of immature cells into particular cell types may create a chimeric polypeptide which may be effective in the treatment of neurological conditions, e.g., nerve damage and neurodegenerative diseases, hyperplastic and neoplastic disorders of pancreatic tissue, and other conditions characterized by undesirable proliferation and differentiation of tissue. Inclusion of a peptide fragment which induces apoptosis may provide a polypeptide effective in treating diseases marked by unwanted cell proliferation, such as cancer, and other conditions known to those in the art as amenable to apoptotic therapy. Inclusion of an anti-angiogenic peptide fragment, e.g., a fragment of angiostatin or endostatin, may yield a chimeric polypeptide useful in the treatment of cancer and other conditions resulting from or enabled by angiogenesis.

Definitions

The term ‘peptide’ refers to an oligomer in which the monomers are amino acids (usually alpha-amino acids) joined together through amide bonds. Peptides are two or more amino acid monomers long, but more often are between 5 to 10 amino acid monomers long and can be even longer, i.e., up to 20 amino acids or more, although peptides longer than 20 amino acids are more likely to be called ‘polypeptides’. The term ‘protein’ is well known in the art and usually refers to a very large polypeptide, or set of associated homologous or heterologous polypeptides, that has some biological function. For purposes of the present invention the terms ‘peptide’, ‘polypeptide’, and ‘protein’ are largely interchangeable as all three types are collectively referred to as peptides.

The interchangeable terms ‘fusion’ and ‘chimeric’, as used herein to describe proteins and polypeptides, relate to polypeptides or proteins wherein two individual polypeptides or portions thereof are fused to form a single amino acid chain. Such fusion may arise from the expression of a single continuous coding sequence formed by recombinant DNA techniques. Thus, ‘fusion’ polypeptides and ‘chimeric’ polypeptides include contiguous polypeptides comprising a first polypeptide covalently linked via an amide bond to one or more amino acid sequences which define polypeptide domains that are foreign to and not substantially homologous with any domain of the first polypeptide.

Gene constructs encoding fusion proteins are likewise referred to a ‘chimeric genes’ or ‘fusion genes’.

‘Homology’ and ‘identity’ each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. An ‘unrelated’, ‘heterologous’, or ‘non-homologous’ sequence shares less than 40 percent identity, though preferably less than 25 percent identity, with a sequence to which it is compared. Thus, a ‘heterologous peptide sequence’ is a peptide sequence substantially dissimilar to a sequence to which it is compared.

The term ‘serum albumin’ (SA) is intended to include (but not necessarily to be restricted to) serum albumin proteins of living organisms, preferably mammalian serum albumins, even more preferably known or yet-to-be-discovered polymorphic forms of human serum albumin (HSA), and variants thereof. For example, the human serum albumin Naskapi has Lys-372 in place of Glu-372, and albumin Christchurch has an altered pro-sequence. The term ‘variants’ is intended to include (but not necessarily be restricted to) homologs of SA proteins with minor artificial variations in sequence (such as molecules lacking one or a few residues, having conservative substitutions or minor insertions of residues, or having minor variations of amino acid structure). Thus, polypeptides which have 80%, 85%, 90%, or 99% homology with a native SA are deemed to be ‘variants’. It is also preferred for such variants to share at least one pharmacological utility with a native SA. Any putative variant which is to be used pharmacologically should be non-immunogenic in the animal (especially human) being treated. Sequences of a number of contemplated serum albumin proteins can be obtained from GenBank (National Center for Biotechnology Information), including human, bovine, mouse, pig, horse, sheep, and chick serum albumins.

The term ‘native’ is used to describe a protein which occurs naturally in a living organism. Wild-type proteins are thus native proteins. Proteins which are non-native are those which have been generated by artificial mutation, recombinant design, or other laboratory modification and are not known in natural populations.

‘Conservative substitutions’ are those where one or more amino acids are substituted for others having similar properties such that one skilled in the art of polypeptide chemistry would expect at least the secondary structure, and preferably the tertiary structure, of the polypeptide to be substantially unchanged. For example, typical such substitutions include asparagine for glutamine, serine for asparagine, and arginine for lysine. The term ‘physiologically functional equivalents’ also encompasses larger molecules comprising the native sequence plus a further sequence at the N-terminus (for example, pro-HSA, pre-pre-HSA, and met-HSA).

‘Tertiary structure’ refers to the three-dimensional structure of a protein. Proteins which have similar tertiary structures will have similar shapes and surfaces, even if the amino acid sequences (the ‘secondary structure’) is not identical. Tertiary structure is a consequence of the folding and twisting of an amino acid chain upon itself and can be disrupted by chemical means, e.g., strong acid or base, or by physical means, e.g., heating.

The term ‘biologically active’ refers to an entity which interacts in some way with a living organism on a molecular level. Entities which are biologically active may activate a receptor, provoke an immune reaction, interact with a membrane or ion channel, or otherwise induce a change in a biological function of an organism or any part of an organism.

The term ‘ligand’ refers to a molecule that is recognized by a particular protein, e.g., a receptor. Any agent bound by or reacting with a protein is called a ‘ligand’, so the term encompasses the substrate of an enzyme and the reactants of a catalyzed reaction. The term ‘ligand’ does not imply any particular molecular size or other structural or compositional feature other than that the substance in question is capable of binding or otherwise interacting with a protein. A ‘ligand’ may serve either as the natural ligand to which the protein binds or as a functional analogue that may act as an agonist or antagonist.

The term ‘vector’ refers to a DNA molecule, capable of replication in a host cell, into which a gene can be inserted to construct a recombinant DNA molecule. Examples of vectors include plasmids and infective microorganisms such as viruses, or non-viral vectors such as ligand-DNA conjugates, liposomes, or lipid-DNA complexes.

As used herein, ‘cell surface receptor’ refers to molecules that occur on the surface of cells, interact with the extracellular environment, and (directly or indirectly) transmit or transduce the information regarding the environment intracellularly in a manner that may modulate intracellular second messenger activities or transcription of specific promoters, resulting in transcription of specific genes.

As used herein, ‘extracellular signals’ include a molecule or other change in the extracellular environment that is transduced intracellularly via cell surface proteins that interact, directly or indirectly, with the signal. An extracellular signal or effector molecule includes any compound or substance that in some manner alters the activity of a cell surface protein. Examples of such signals include, but are not limited to, molecules such as acetylcholine, growth factors and hormones, lipids, sugars and nucleotides that bind to cell surface and/or intracellular receptors and ion channels and modulate the activity of such receptors and channels.

As used herein, ‘extracellular signals’ also include as yet unidentified substances that modulate the activity of a cellular receptor, and thereby influence intracellular functions. Such extracellular signals are potential pharmacological agents that may be used to treat specific diseases by modulating the activity of specific cell surface receptors.

‘Orphan receptors’ is a designation given to receptors for which no specific natural ligand has been described and/or for which no function has been determined.

The term ‘target cells’ as used herein means cells, either in vivo or ex vivo, into which it is desired to introduce exogenous genetic material. Target cells may be any type of cell, including blood cells, skeletal muscle cells, stem cells, skin cells, liver cells, secretory gland cells, hematopoietic cells, and marrow cells.

An ‘effective amount’ of a fusion polypeptide, with respect to the subject method of treatment, refers to an amount of the polypeptide in a preparation which, when applied as part of a desired dosage regimen, provides inhibition of angiogenesis so as to reduce or cure a disorder according to clinically acceptable standards.

‘Serum half-life’ as used herein refers to the time required for half of a quantity of a peptide in the bloodstream to be degraded.

Exemplification

As set out above, the chimeric polypeptide of the present invention can be constructed as a chimeric polypeptide containing a sequence homologous to at least a portion of a serum albumin and at least a portion of one or more heterologous proteins, expressed as one contiguous polypeptide chain. In preparing the chimeric polypeptide, a fusion gene is constructed comprising DNA encoding at least one sequence each of a serum albumin, a heterologous protein, and, optionally, a peptide linker sequence to span the fragments. If more than one heterologous sequences are included in the chimeric polypeptide, they may be identical, related, or unrelated sequences. Identical sequences may be included to increase the effective concentration of the sequence. Related sequences may be included to more accurately mimic the native protein from which they are derived. Unrelated sequences may be useful for activating two or more distinct receptors that stimulate the same response, or for imparting two or more distinct activities to the chimeric polypeptide. For example, the chimeric polypeptide might include a sequence that has antiangiogenic activity and a sequence which induces apoptosis of tumor cells.

To make this chimeric polypeptide, an entire protein can be cloned and expressed as part of the protein, or alternatively, a suitable fragment thereof containing a biologically active moiety can be used. The use of recombinant DNA techniques to create a fusion gene, with the translational product being the desired chimeric polypeptide, is well known in the art. Both the coding sequence of a gene and its regulatory regions can be redesigned to change the functional properties of the protein product, the amount of protein made, or the cell type in which the protein is produced. The coding sequence of a gene can be extensively altered, for example, by fusing part of it to the coding sequence of a different gene to produce a novel hybrid gene that encodes a fusion protein. Examples of methods for producing fusion proteins are described in PCT applications PCT/US87/02968, PCT/US89/03587 and PCT/US90/07335, as well as Traunecker et al. (1989) Nature 339:68, all of which are incorporated by reference herein.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992).

This invention also provides expression vectors comprising a nucleotide sequence encoding a subject chimeric polypeptide operably linked to at least one regulatory sequence. ‘Operably linked’ is intended to mean that the nucleotide sequence is linked to a regulatory sequence in a manner which allows expression of the nucleotide sequence. Regulatory sequences are art-recognized and are selected to direct expression of the encoded polypeptide. Accordingly, the term regulatory sequence includes promoters, enhancers and other expression control elements. Exemplary regulatory sequences are described in Goeddel; Gene Expression Technology: Methods in Enzymology 185, Academic Press, San Diego, Calif. (1990). For instance, any of a wide variety of expression control sequences-sequences that control the expression of a DNA sequence when operatively linked to it may be used in these vectors to express DNA sequences encoding the chimeric polypeptides of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

As will be apparent, the subject gene constructs can be used to cause expression of the subject chimeric polypeptides in cells propagated in culture, e.g., to produce chimeric polypeptides, for purification. This represents a method for preparing substantial quantities of the polypeptide, e.g., for research, clinical, and pharmaceutical uses.

In certain therapeutic applications, the ex vivo-derived chimeric polypeptides are utilized in a manner appropriate for therapy in general. For such therapy, the polypeptides of the invention can be formulated for a variety of modes of administration, including systemic and topical or localized administration. In such embodiments, the polypeptide may by combined with a pharmaceutically acceptable excipient, e.g., a non-pyrogenic excipient. Techniques and formulations generally may be found in Remmington's Pharmaceutical Sciences, Meade Publishing Co., Easton, Pa. For systemic administration, injection being preferred, including intramuscular, intravenous, intraperitoneal, and subcutaneous injection, the polypeptides of the invention can be formulated in liquid solutions, preferably in physiologically compatible buffers such as Hank's solution or Ringer's solution. In addition, the polypeptides may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the peptides are formulated into conventional oral administration forms such as capsules, tablets, and tonics. For topical administration, particularly cosmetic formulations, the oligomers of the invention are formulated into ointments, salves, gels, or creams as generally known in the art.

Alternative means of administration of peptides have been developed. Sustained-release formulations (Putney, et al. Nature Biotechnology 1998, 16, 153-157) are advantageous, requiring fewer administrations and, often, lower dosages. Techniques for oral delivery of peptides have been reviewed (Fasano, A. Trends in Biotechnology 1998, 16, 152-157), as have several site-specific means of peptide delivery (Pettit, D. K. et al. Trends in Biotechnology 1998, 16, 343-349). Additional techniques for therapeutic administration of peptides are known to those of skill in the art.

Genetic material of the present invention can be delivered, for example, as an expression plasmid which, when transcribed in the cell, produces the desired chimeric polypeptide.

In another embodiment, the genetic material is provided by use of an “expression” construct, which can be transcribed in a cell to produce the chimeric polypeptide. Such expression constructs may be administered in any biologically effective carrier, e.g., any formulation or composition capable of effectively transfecting cells either ex vivo or in vivo with genetic material encoding a chimeric polypeptide. Approaches include insertion of the antisense nucleic acid in viral vectors including recombinant retroviruses, adenoviruses, adeno-associated viruses, human immunodeficiency viruses, and herpes simplex viruses-1, or recombinant bacterial or eukaryotic plasmids. Viral vectors can be used to transfect cells directly; plasmid DNA can be delivered with the help of, for example, cationic liposomes (lipofectin) or derivatized (e.g., antibody conjugated), polylysine conjugates, gramacidin S, artificial viral envelopes or other such intracellular carriers, as well as direct injection of the gene construct or calcium phosphate precipitation carried out in vivo. It will be appreciated that because transduction of appropriate target cells represents the critical first step in gene therapy, choice of the particular gene delivery system will depend on such factors as the phenotype of the intended target and the route of administration, e.g., locally or systemically.

A preferred approach for in vivo introduction of genetic material encoding one of the subject proteins into a cell is by use of a viral vector containing said genetic material. Infection of cells with a viral vector has the advantage that a large proportion of the targeted cells can receive the nucleic acid. Additionally, chimeric polypeptides encoded by genetic material in the viral vector, e.g., by a nucleic acid contained in the viral vector, are expressed efficiently in cells which have taken up viral vector nucleic acid. Such a strategy may be particularly effective when skeletal muscle cells are the targets of the vector (Fisher, K. J. et al. Nature Medicine 1997, 3, 306-312).

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding one of the antisense E6AP constructs, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing retroviral vectors as a gene delivery system for genetic material encoding the subject chimeric polypeptides, it is important to note that a prerequisite for the successful infection of target cells by most retroviruses, and therefore of stable introduction of the genetic material, is that the target cells must be dividing. In general, this requirement will not be a hindrance to use of retroviral vectors. In fact, such limitation on infection can be beneficial in circumstances wherein the tissue (e.g., nontransformed cells) surrounding the target cells does not undergo extensive cell division and is therefore refractory to infection with retroviral vectors.

Furthermore, it has been shown that it is possible to limit the infection spectrum of retroviruses and consequently of retroviral-based vectors, by modifying the viral packaging proteins on the surface of the viral particle (see, for example, PCT publications WO93/25234, WO94/06920, and WO94/11524). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface ligands to the viral env proteins (Neda et al. (1991) J Biol Chem 266:14143-14146). Coupling can be in the form of the chemical cross-linking with a protein or other variety (e.g., lactose to convert the env protein to an asialoglycoprotein), as well as by generating chimeric proteins (e.g., single-chain antibody/env chimeric proteins). This technique, while useful to limit or otherwise direct the infection to certain tissue types, and can also be used to convert an ecotropic vector in to an amphotropic vector.

Moreover, use of retroviral gene delivery can be further enhanced by the use of tissue- or cell-specific transcriptional regulatory sequences which control expression of the genetic material of the retroviral vector.

Another viral gene delivery system useful in the present invention utilizes adenovirus-derived vectors. The genome of an adenovirus can be manipulated such that it encodes a gene product of interest, but is inactive in terms of its ability to replicate in a normal lytic viral life cycle (see, for example, Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 dl324 or other strains of adenovirus (e.g., Ad2, Ad3, Ad7, etc.) are well known to those skilled in the art. Recombinant adenoviruses can be advantageous in certain circumstances in that they are capable of infecting non-dividing cells and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Furthermore, the virus particle is relatively stable and amenable to purification and concentration, and, as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, for example, Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted genetic material can be under control of, for example, the E1A promoter, the major late promoter (MLP) and associated leader sequences, the E3 promoter, or exogenously added promoter sequences.

Yet another viral vector system useful for delivery of genetic material encoding the subject chimeric polypeptides is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

Other viral vector systems that may have application in gene therapy have been derived from herpes virus, vaccinia virus, and several RNA viruses.

In addition to viral transfer methods, such as those illustrated above, non-viral methods can also be employed to cause expression of genetic material encoding the subject chimeric polypeptides in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of genetic material by the targeted cell. Exemplary gene delivery systems of this type include liposomal derived systems, polylysine conjugates, and artificial viral envelopes.

In a representative embodiment, genetic material can be entrapped in liposomes bearing positive charges on their surface (e.g., lipofectins) and, optionally, which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, lipofection of papilloma-infected cells can be carried out using liposomes tagged with monoclonal antibodies against PV-associated antigen (see Viac et al. (1978) J Invest Dermatol 70:263-266; see also Mizuno et al. (1992) Neurol. Med. Chir. 32:873-876).

In yet another illustrative embodiment, the gene delivery system comprises an antibody or cell surface ligand which is cross-linked with a gene binding agent such as polylysine (see, for example, PCT publications WO93/04701, WO92/22635, WO92/20316, WO92/19749, and WO92/06180). For example, genetic material encoding the subject chimeric polypeptides can be used to transfect hepatocytic cells in vivo using a soluble polynucleotide carrier comprising an asialoglycoprotein conjugated to a polycation, e.g., polylysine (see U.S. Pat. No. 5,166,320). It will also be appreciated that effective delivery of the subject nucleic acid constructs via mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al. (1993) Science 260-926; Wagner et al. (1992) PNAS 89:7934; and Christiano et al. (1993) PNAS 90:2122).

In clinical settings, the gene delivery systems can be introduced into a patient by any of a number of methods, each of which is familiar in the art. For instance, a pharmaceutical preparation of the gene delivery system can be introduced systemically, e.g., by intravenous injection, and specific transduction of the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057).

Moreover, the pharmaceutical preparation can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral packages, the pharmaceutical preparation can comprise one or more cells which produce the gene delivery system. In the latter case, methods of introducing the viral packaging cells may be provided by, for example, rechargeable or biodegradable devices. Various slow release polymeric devices have been developed and tested in vivo in recent years for the controlled delivery of drugs, including proteinaceous biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of an the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art. See, for example, Concise Encyclopedia of Medical & Dental Materials, ed. by David Williams (MIT Press: Cambridge, Mass., 1990); and the Sabel et al. U.S. Pat. No. 4,883,666. In another embodiment of an implant, a source of cells producing a the recombinant virus is encapsulated in implantable hollow fibers. Such fibers can be pre-spun and subsequently loaded with the viral source (Aebischer et al. U.S. Pat. No. 4,892,538; Aebischer et al. U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183), or can be co-extruded with a polymer which acts to form a polymeric coat about the viral packaging cells (Lim U.S. Pat. No. 4,391,909; Sefton U.S. Pat. No. 4,353,888; Sugamori et al. (1989) Trans. Am. Artif. Intern. Organs 35:791-799; Sefton et al. (1987) Biotechnol. Bioeng. 29:1135-1143; and Aebischer et al. (1991) Biomaterials 12:50-55). Again, manipulation of the polymer can be carried out to provide for optimal release of viral particles.

Chimeric polypeptides of the present invention can be designed by using molecular modeling. A computer model of serum albumin may be altered to include a selected heterologous sequence and the resulting structure may be submitted to calculations designed to determine how the resulting peptide will change in shape, how much strain the alteration introduces into the polypeptide, how the heterologous sequence is displayed in three dimensions, and other data relevant to the resulting structure of the chimeric polypeptide. Alternatively, the nature of the sequence to be included might be determined by the calculation, based on knowledge of a receptor or binding pocket. In another embodiment, the calculations might best determine how to insert a desired sequence to maintain the tertiary structure of the serum albumin backbone, or to display the insertion in the proper orientation. Other calculational strategies will be known to those skilled in the art. Calculations such as these can be useful for directing the synthesis of chimeric polypeptides of the present invention in a time- and material-efficient manner, before actual synthesis and screening techniques begin.

Methods for screening chimeric polypeptides of the present invention are well known in the art, independent of the use of computer modeling. The use of peptide libraries is one way of screening large numbers of polypeptides at once. In one screening assay, the candidate peptides are displayed on the surface of a cell or viral particle, and the ability of particular cells or viral particles to bind a target molecule, such as a receptor protein via this gene product is detected in a “panning assay”. For instance, the gene library can be cloned into the gene for a surface membrane protein of a bacterial cell, and the resulting chimeric polypeptide detected by panning (Ladner et al., WO 88/06630; Fuchs et al. (1991) Bio/Technology 9:1370-1371; and Goward et al. (1992) TIBS 18:136-140).

In an alternate embodiment, the peptide library is expressed as chimeric polypeptides on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate chimeric polypeptides without disrupting the ultimate packaging of the viral particle (Ladner et al. PCT publication WO 90/02809; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

The field of combinatorial peptide libraries has been reviewed (Gallop et al. J. Med. Chem. 1994, 37, 1233-1251), and additional techniques are known in the art (Gustin, K. Virology 1993, 193, 653-660; Goeddel et al. U.S. Pat. No. 5,223,408; Markland et al. PCT publication WO92/15679; Bass et al. Proteins: Structure, Function and Genetics 1990, 8, 309-314;Cunningham, B. C. Science 1990, 247, 1461-1465; Lowman, H. B. Biochemistry 1991, 30, 10832-10838; Fowlkes et al. U.S. Pat. No. 5,789,184; Houghton, Proc. Natl. Acad. Sci. U.S.A. 1985, 82, 5131-5135) for generating and screening peptide libraries.

U.S. patent application Ser. No. 09/174,943, filed Oct. 19, 1998, discloses a method for isolating biologically active peptides. Using the techniques disclosed therein, a chimeric polypeptide of the present invention may be developed which interacts with a chosen receptor.

In a representative example, this method is utilized to identify polypeptides which have antiproliferative activity with respect to one or more types of cells. One of skill in the art will readily be able to modify the procedures outlined below to find polypeptides with any desired activity. In the example, in the display mode, the chimeric polypeptide library can be panned with the target cells for which an antiproliferative is desired in order to enrich for polypeptides which bind to that cell. At that stage, the polypeptide library can also be panned against one or more control cell lines in order to remove polypeptides which bind the control cells. In this manner, the polypeptide library which is then tested in the secretion mode can be enriched for polypeptides which selectively bind target cells (relative to the control cells). Thus, for example, the display mode can produce a polypeptide library enriched for polypeptides which preferentially bind tumor cells relative to normal cells, which preferentially bind p53− cells relative to p53+ cells, which preferentially bind hair follicle cells relative to other epithelial cells, or any other differential binding characteristic.

In the secretion mode, the polypeptides are tested for antiproliferative activity against the target cell using any of a number of techniques known in the art. For instance, BrdU or other nucleotide uptake can be measured as an indicator of proliferation. As above, the secretion mode can include negative controls in order to select for polypeptides with specific antiproliferative activity.

In similar fashion, polypeptides can be isolated from the library based on their ability to induce apoptosis or cell lysis, for example, in a cell-selective manner.

Also, this method can be used to identify polypeptides with angiogenic or antiangiogenic activity. For instance, the polypeptide library can be enriched for polypeptides that bind to endothelial cells but which do not bind to fibroblasts. The resulting sub-library can be screened for polypeptides which inhibit capillary endothelial cell proliferation and/or endothelial cell migration. Polypeptides scoring positive for one or both of these activities can also be tested for activity against other cell types, such as smooth muscle cells or fibroblasts, in order to select polypeptides active only against endothelial cells.

Furthermore, this method can be used to identify anti-infective polypeptides, for example, which are active as anti-fungal or antibacterial agents.

In addition, this assay can be used for identifying effectors of a receptor protein or complex thereof. In general, the assay is characterized by the use of a test cell which includes a target receptor or ion channel protein whose signal transduction activity can be modulated by interaction with an extracellular signal, the transduction activity being able to generate a detectable signal.

In general, such assays are characterized by the use of a mixture of cells expressing a target receptor protein or ion channel capable of transducing a detectable signal in the reagent cell. The receptor/channel protein can be either endogenous or heterologous. In combination with the disclosed detection means, a culture of the instant reagent cells will provide means for detecting agonists or antagonists of receptor function.

The ability of particular polypeptides to modulate a signal transduction activity of the target receptor or channel can be scored for by detecting up or down-regulation of the detection signal. For example, second messenger generation (e.g., GTPase activity, phospholipid hydrolysis, or protein phosphorylation patterns as examples) can be measured directly. Alternatively, the use of an indicator gene can provide a convenient readout. In other embodiments a detection means consists of an indicator gene. In any event, a statistically significant change in the detection signal can be used to facilitate identification of compounds which modulate receptor or ion channel activities.

By this method, polypeptides which induce a signal pathway from a particular receptor or channel can be identified. If a test polypeptide does not appear to induce the activity of the receptor/channel protein, the assay may be repeated as described above, and modified by the introduction of a step in which the reagent cell is first contacted with a known activator of the target receptor/channel to induce signal transduction, and the test peptide can be assayed for its ability to inhibit the activated receptor/channel, for example, to identify antagonists. In yet other embodiments, peptides can be screened for those which potentiate the response to a known activator of the receptor.

In particular, the assays can be used to test functional ligand-receptor or ligand-ion channel interactions for cell surface-localized receptors and channels. As described in more detail below, the subject assay can be used to identify effectors of, for example, G protein-coupled receptors, receptor tyrosine kinases, cytokine receptors, and ion channels. In certain embodiments the method described herein is used for identifying ligands for “orphan receptors” for which no ligand is known.

In some examples, the receptor is a cell surface receptor, such as: a receptor tyrosine kinase, for example, an EPH receptor; an ion channel; a cytokine receptor; an multisubunit immune recognition receptor, a chemokine receptor; a growth factor receptor, or a G-protein coupled receptor, such as a chemoattracttractant peptide receptor, a neuropeptide receptor, a light receptor, a neurotransmitter receptor, or a polypeptide hormone receptor.

Preferred G protein-coupled receptors include α1A-adrenergic receptor, α1B-adrenergic receptor, α2-adrenergic receptor, α2B-adrenergic receptor, 1-adrenergic receptor, β2-adrenergic receptor, β3-adrenergic receptor, m1 acetylcholine receptor (AChR), m2 AChR, m3 AChR, m4 AChR, m5 AChR, D1 dopamine receptor, D2 dopamine receptor, D3 dopamine receptor, D4 dopamine receptor, D5 dopamine receptor, A1 adenosine receptor, A2b adenosine receptor, 5-HT1a receptor, 5-HT1b receptor, 5HT1-like receptor, 5-HT1d receptor, 5HT1d-like receptor, 5HT1d beta receptor, substance K (neurokinin A) receptor, fMLP receptor, fMLP-like receptor, angiotensin II type 1 receptor, endothelin ETA receptor, endothelin ETB receptor, thrombin receptor, growth hormone-releasing hormone (GHRH) receptor, vasoactive intestinal peptide receptor, oxytocin receptor, somatostatin SSTR1 and SSTR2, SSTR3, cannabinoid receptor, follicle stimulating hormone (FSH) receptor, leutropin (LH/HCG) receptor, thyroid stimulating hormone (TSH) receptor, thromboxane A2 receptor, platelet-activating factor (PAF) receptor, C5a anaphylatoxin receptor, Interleukin 8 (IL-8) IL-8RA, IL-8RB, Delta Opioid receptor, Kappa Opioid receptor, mip-1/RANTES receptor, Rhodopsin, Red opsin, Green opsin, Blue opsin, metabotropic glutamate mGluR1-6, histamine H2 receptor, ATP receptor, neuropeptide Y receptor, amyloid protein precursor receptor, insulin-like growth factor II receptor, bradykinin receptor, gonadotropin-releasing hormone receptor, cholecystokinin receptor, melanocyte stimulating hormone receptor receptor, antidiuretic hormone receptor, glucagon receptor, and adrenocorticotropic hormone II receptor.

Preferred EPH receptors inlcude eph, elk, eck, sek, mek4, hek, hek2, eek, erk, tyrol, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors.

A. Cytokine Receptors

In one example the target receptor is a cytokine receptor. Cytokines are a family of soluble mediators of cell-to-cell communication that includes interleukins, interferons, and colony-stimulating factors. The characteristic features of cytokines lie in their functional redundancy and pleiotropy. Most of the cytokine receptors that constitute distinct superfamilies do not possess intrinsic protein tyrosine kinase domains, yet receptor stimulation usually invokes rapid tyrosine phosphorylation of intracellular proteins, including the receptors themselves. Many members of the cytokine receptor superfamily activate the Jak protein tyrosine kinase family, with resultant phosphorylation of the STAT transcriptional activator factors. IL-2, IL-7, IL-2 and Interferon γ have all been shown to activate Jak kinases (Frank et al (1995) Proc Natl Acad Sci USA 92:7779-7783); Scharfe et al. (1995) Blood 86:2077-2085); (Bacon et al. (1995) Proc Natl Acad Sci USA 92:7307-7311); and (Sakatsume et al (1995) J. Biol Chem 270:17528-17534). Events downstream of Jak phosphorylation have also been elucidated. For example, exposure of T lymphocytes to IL-2 has been shown to lead to the phosphorylation of signal transducers and activators of transcription (STAT) proteins STAT1α, STAT2β, and STAT3, as well as of two STAT-related proteins, p94 and p95. The STAT proteins were found to translocate to the nucleus and to bind to a specific DNA sequence, thus suggesting a mechanism by which IL-2 may activate specific genes involved in immune cell function (Frank et al. supra). Jak3 is associated with the gamma chain of the IL-2 , IL-4, and IL-7 cytokine receptors (Fujii et al. (1995) Proc Natl Acad Sci 92:5482-5486) and (Musso et al (1995) J Exp Med. 181:1425-1431). The Jak kinases have also been shown to be activated by numerous ligands that signal via cytokine receptors such as, growth hormone and erythropoietin and IL-6 (Kishimoto (1994) Stem cells Suppl 12:37-44).

Detection means which may be scored for in the present assay, in addition to direct detection of second messengers, such as by changes in phosphorylation, includes reporter constructs or indicator genes which include transcriptional regulatory elements responsive to the STAT proteins. Described infra.

B Multisubunit Immune Recognition Receptor (MIRR)

In another example the receptor is a multisubunit receptor. Receptors can be comprised of multiple proteins referred to as subunits, one category of which is referred to as a multisubunit receptor is a multisubunit immune recognition receptor (MIRR). MIRRs include receptors having multiple noncovalently associated subunits and are capable of interacting with src-family tyrosine kinases. MIRRs can include, but are not limited to, B cell antigen receptors, T cell antigen receptors, Fc receptors and CD22. One example of an MIRR is an antigen receptor on the surface of a B cell. To further illustrate, the MIRR on the surface of a B cell comprises membrane-bound immunoglobulin (mIg) associated with the subunits Ig-α and Ig- or Ig-γ, which forms a complex capable of regulating B cell function when bound by antigen. An antigen receptor can be functionally linked to an amplifier molecule in a manner such that the amplifier molecule is capable of regulating gene transcription.

Src-family tyrosine kinases are enzymes capable of phosphorylating tyrosine residues of a target molecule. Typically, a src-family tyrosine kinase contains one or more binding domains and a kinase domain. A binding domain of a src-family tyrosine kinase is capable of binding to a target molecule and a kinase domain is capable of phosphorylating a target molecule bound to the kinase. Members of the src family of tyrosine kinases are characterized by an N-terminal unique region followed by three regions that contain different degrees of homology among all the members of the family. These three regions are referred to as src homology region 1 (SH1), src homology region 2 (SH2) and src homology region 3 (SH3). Both the SH2 and SH3 domains are believed to have protein association functions important for the formation of signal transduction complexes. The amino acid sequence of an N-terminal unique region, varies between each src-family tyrosine kinase. An N-terminal unique region can be at least about the first 40 amino acid residues of the N-terminal of a src-family tyrosine kinase.

Syk-family kinases are enzymes capable of phosphorylating tyrosine residues of a target molecule. Typically, a syk-family kinase contains one or more binding domains and a kinase domain. A binding domain of a syk-family tyrosine kinase is capable of binding to a target molecule and a kinase domain is capable of phosphorylating a target molecule bound to the kinase. Members of the syk family of tyrosine kinases are characterized by two SH2 domains for protein association function and a tyrosine kinase domain.

A primary target molecule is capable of further extending a signal transduction pathway by modifying a second messenger molecule. Primary target molecules can include, but are not limited to, phosphatidylinositol 3-kinase (PI-3K), P21^{ras}GAPase-activating protein and associated P190 and P62 protein, phospholipases such as PLCγ1 and PLC2, MAP kinase, Shc and VAV. A primary target molecule is capable of producing second messenger molecule which is capable of further amplifying a transduced signal. Second messenger molecules include, but are not limited to diacylglycerol and inositol 1,4,5-triphosphate (IP3). Second messenger molecules are capable of initiating physiological events which can lead to alterations in gene transcription. For example, production of IP3 can result in release of intracellular calcium, which can then lead to activation of calmodulin kinase II, which can then lead to serine phosphorylation of a DNA binding protein referred to as ets-1 proto-onco-protein. Diacylglycerol is capable of activating the signal transduction protein, protein kinase C which affects the activity of the AP1 DNA binding protein complex. Signal transduction pathways can lead to transcriptional activation of genes such as c-fos, egr-1, and c-myc.

Shc can be thought of as an adaptor molecule. An adaptor molecule comprises a protein that enables two other proteins to form a complex (e.g., a three molecule complex). Shc protein enables a complex to form which includes Grb2 and SOS. Shc comprises an SH2 domain that is capable of associating with the SH2 domain of Grb2.

Molecules of a signal transduction pathway can associate with one another using recognition sequences. Recognition sequences enable specific binding between two molecules. Recognition sequences can vary depending upon the structure of the molecules that are associating with one another. A molecule can have one or more recognition sequences, and as such can associate with one or more different molecules.

Signal transduction pathways for MIRR complexes are capable of regulating the biological functions of a cell. Such functions can include, but are not limited to the ability of a cell to grow, to differentiate and to secrete cellular products. MIRR-induced signal transduction pathways can regulate the biological functions of specific types of cells involved in particular responses by an animal, such as immune responses, inflammatory responses and allergic responses. Cells involved in an immune response can include, for example, B cells, T cells, macrophages, dendritic cells, natural killer cells and plasma cells. Cells involved in inflammatory responses can include, for example, basophils, mast cells, eosinophils, neutrophils and macrophages. Cells involved in allergic responses can include, for example mast cells, basophils, B cells, T cells and macrophages.

In certain examples, the detection signal is a second messenger, such as a phosphorylated src-like protein, including reporter constructs or indicator genes which include transcriptional regulatory elements such as serum response element (SRE), 12-O-tetradecanoyl-phorbol-13-acetate response element, cyclic AMP response element, c-fos promoter, or a CREB-responsive element.

C. Receptor Tyrosine Kinases

In still another example, the target receptor is a receptor tyrosine kinase. The receptor tyrosine kinases can be divided into five subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the eph/eck family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence ( Hanks et al. (1988) Science 241:42-52). Subgroups III (platelet-derived growth factor (PDGF) receptor-like) and IV (the fibro-blast growth factor (FGF) receptors) are characterized as having immunoglobulin (Ig)-like folds in their extracellular domains, as well as having their kinase domains divided in two parts by a variable stretch of unrelated amino acids (Yanden and Ullrich (1988) supra and Hanks et al. (1988) supra).

The family with by far the largest number of known members is the EPH family. Since the description of the prototype, the EPH receptor (Hirai et al. (1987) Science 238:1717-1720), sequences have been reported for at least ten members of this family, not counting apparently orthologous receptors found in more than one species. Additional partial sequences, and the rate at which new members are still being reported, suggest the family is even larger (Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br J Cancer 69:417-421). Remarkably, despite the large number of members in the EPH family, all of these molecules were identified as orphan receptors without known ligands.

The expression patterns determined for some of the EPH family receptors have implied important roles for these molecules in early vertebrate development. In particular, the timing and pattern of expression of sek, mek4 and some of the other receptors during the phase of gastrulation and early organogenesis has suggested functions for these receptors in the important cellular interactions involved in patterning the embryo at this stage (Gilardi-Hebenstreit et al. (1992) Oncogene 7:2499-2506; Nieto et al. (1992) Development 116:1137-1150; Henkemeyer et al., supra; Ruiz et al., supra; and Xu et al., supra). Sek, for example, shows a notable early expression in the two areas of the mouse embryo that show obvious segmentation, namely the somites in the mesoderm and the rhombomeres of the hindbrain; hence the name sek, for segmentally expressed kinase (Gilardi-Hebenstreit et al., supra; Nieto et al., supra). As in Drosophila, these segmental structures of the mammalian embryo are implicated as important elements in establishing the body plan. The observation that Sek expression precedes the appearance of morphological segmentation suggests a role for sek in forming these segmental structures, or in determining segment-specific cell properties such as lineage compartmentation (Nieto et al., supra). Moreover, EPH receptors have been implicated, by their pattern of expression, in the development and maintenance of nearly every tissue in the embryonic and adult body. For instance, EPH receptors have been detected throughout the nervous system, the testes, the cartilaginous model of the skeleton, tooth primordia, the infundibular component of the pituitary, various epithelial tissues, lung, pancreas, liver and kidney tissues. Observations such as this have been indicative of important and unique roles for EPH family kinases in development and physiology, but further progress in understanding their action has been severely limited by the lack of information on their ligands.

As used herein, the terms “EPH receptor” or “EPH-type receptor” refer to a class of receptor tyrosine kinases, comprising at least eleven paralogous genes, though many more orthologs exist within this class, e.g., homologs from different species. EPH receptors, in general, are a discrete group of receptors related by homology and easily recognizable, for example, they are typically characterized by an extracellular domain containing a characteristic spacing of cysteine residues near the N-terminus and two fibronectin type III repeats (Hirai et al. (1987) Science 238:1717-1720; Lindberg et al. (1990) Mol Cell Biol 10:6316-6324; Chan et al. (1991) Oncogene 6:1057-1061; Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br J Cancer 69:417-421). Exemplary EPH receptors include the eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. The term “EPH receptor” refers to the membrane form of the receptor protein, as well as soluble extracellular fragments which retain the ability to bind the ligand of the present invention.

In certain examples, the detection signal is provided by detecting phosphorylation of intracellular proteins, e.g., MEKKs, MEKs, or Map kinases, or by the use of reporter constructs or indicator genes which include transcriptional regulatory elements responsive to c-fos and/or c-jun. Described infra.

D. G Protein-Coupled Receptors

One family of signal transduction cascades found in eukaryotic cells utilizes heterotrimeric “G proteins.” Many different G proteins are known to interact with receptors. G protein signaling systems include three components: the receptor itself, a GTP-binding protein (G protein), and an intracellular target protein.

The cell membrane acts as a switchboard. Messages arriving through different receptors can produce a single effect if the receptors act on the same type of G protein. On the other hand, signals activating a single receptor can produce more than one effect if the receptor acts on different kinds of G proteins, or if the G proteins can act on different effectors.

In their resting state, the G proteins, which consist of alpha (α), beta (β) and gamma (γ) subunits, are complexed with the nucleotide guanosine diphosphate (GDP) and are in contact with receptors. When a hormone or other first messenger binds to receptor, the receptor changes conformation and this alters its interaction with the G protein. This spurs the α subunit to release GDP, and the more abundant nucleotide guanosine triphosphate (GTP), replaces it, activating the G protein. The G protein then dissociates to separate the α subunit from the still complexed beta and gamma subunits. Either the Gα subunit, or the Gβγ complex, depending on the pathway, interacts with an effector. The effector (which is often an enzyme) in turn converts an inactive precursor molecule into an active “second messenger,” which may diffuse through the cytoplasm, triggering a metabolic cascade. After a few seconds, the Gα converts the GTP to GDP, thereby inactivating itself. The inactivated Gα may then reassociate with the Gβγ complex.

Hundreds, if not thousands, of receptors convey messages through heterotrimeric G proteins, of which at least 17 distinct forms have been isolated. Although the greatest variability has been seen in the α subunit, several different β and γ structures have been reported. There are, additionally, several different G protein-dependent effectors.

Most G protein-coupled receptors are comprised of a single protein chain that is threaded through the plasma membrane seven times. Such receptors are often referred to as seven-transmembrane receptors (STRs). More than a hundred different STRs have been found, including many distinct receptors that bind the same ligand, and there are likely many more STRs awaiting discovery.

In addition, STRs have been identified for which the natural ligands are unknown; these receptors are termed “orphan” G protein-coupled receptors, as described above. Examples include receptors cloned by Neote et al. (1993) Cell 72, 415; Kouba et al. FEBS Lett. (1993) 321, 173; Birkenbach et al.(1993) J. Virol. 67, 2209.

The ‘exogenous receptors’ of this example may be any G protein-coupled receptor which is exogenous to the cell which is to be genetically engineered for the purpose of the present invention. This receptor may be a plant or animal cell receptor. Screening for binding to plant cell receptors may be useful in the development of, for example, herbicides. In the case of an animal receptor, it may be of invertebrate or vertebrate origin. If an invertebrate receptor, an insect receptor is preferred, and would facilitate development of insecticides. The receptor may also be a vertebrate, more preferably a mammalian, still more preferably a human, receptor. The exogenous receptor is also preferably a seven transmembrane segment receptor.

Known ligands for G protein coupled receptors include: purines and nucleotides, such as adenosine, cAMP, ATP, UTP, ADP, melatonin and the like; biogenic amines (and related natural ligands), such as 5-hydroxytryptamine, acetylcholine, dopamine, adrenaline, adrenaline, adrenaline., histamine, noradrenaline, noradrenaline, noradrenaline., tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, beta-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheremones, galanin, gastric inhibitory polypeptide receptor (gip), glucagon-like peptides (glps), glucagon, gonadotropin releasing hormone (gnrh), growth hormone releasing hormone(ghrh), insect diuretic hormone, interleukin-8, leutropin (lh/hcg), met-enkephalin, opioid peptides, oxytocin, parathyroid hormone (pth) and pthrp, pituitary adenylyl cyclase activiating peptide (pacap), secretin, somatostatin, thrombin, thyrotropin (tsh), vasoactive intestinal peptide (vip), vasopressin, vasotocin; eicosanoids such as ip-prostacyclin, pg-prostaglandins, tx-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

Suitable examples of G-protein coupled receptors include, but are not limited to, dopaminergic, muscarinic cholinergic, a-adrenergic, b-adrenergic, opioid (including delta and mu), cannabinoid, serotoninergic, and GABAergic receptors. Preferred receptors include the 5HT family of receptors, dopamine receptors, C5a receptor and FPRL-1 receptor, cyclo-histidyl-proline-diketoplperazine receptors, melanocyte stimulating hormone release inhibiting factor receptor, and receptors for neurotensin, thyrotropin releasing hormone, calcitonin, cholecytokinin-A, neurokinin-2, histamine-3, cannabinoid, melanocortin, or adrenomodulin, neuropeptide-Y1 or galanin. Other suitable receptors are listed in the art. The term ‘receptor,’ as used herein, encompasses both naturally occurring and mutant receptors.

Many of these G protein-coupled receptors, like the yeast a- and α-factor receptors, contain seven hydrophobic amino acid-rich regions which are assumed to lie within the plasma membrane. Specific human G protein-coupled STRs for which genes have been isolated and for which expression vectors could be constructed include those listed herein and others known in the art. Thus, the gene would be operably linked to a promoter functional in the cell to be engineered and to a signal sequence that also functions in the cell. For example in the case of yeast, suitable promoters include Ste2, Ste3 and gal10. Suitable signal sequences include those of Ste2, Ste3 and of other genes which encode proteins secreted by yeast cells. Preferably, when a yeast cell is used, the codons of the gene would be optimized for expression in yeast. See Hoekema et al.,(1987) Mol. Cell. Biol., 7:2914-24; Sharp, et al., (1986)14:5125-43.

The homology of STRs is discussed in Dohlman et al., Ann. Rev. Biochem., (1991) 60:653-88. When STRs are compared, a distinct spatial pattern of homology is discernible. The transmembrane domains are often the most similar, whereas the N- and C-terminal regions, and the cytoplasmic loop connecting transmembrane segments V and VI are more divergent.

The functional significance of different STR regions has been studied by introducing point mutations (both substitutions and deletions) and by constructing chimeras of different but related STRs. Synthetic peptides corresponding to individual segments have also been tested for activity. Affinity labeling has been used to identify ligand binding sites.

It is conceivable that when the host cell is a yeast cell, a foreign receptor will fail to functionally integrate into the yeast membrane, and there interact with the endogenous yeast G protein. More likely, either the receptor will need to be modified (e.g., by replacing its V-VI loop with that of the yeast STE2 or STE3 receptor), or a compatible G protein should be provided.

If the wild-type exogenous G protein-coupled receptor cannot be made functional in yeast, it may be mutated for this purpose. A comparison would be made of the amino acid sequences of the exogenous receptor and of the yeast receptors, and regions of high and low homology identified. Trial mutations would then be made to distinguish regions involved in ligand or G protein binding, from those necessary for functional integration in the membrane. The exogenous receptor would then be mutated in the latter region to more closely resemble the yeast receptor, until functional integration was achieved. If this were insufficient to achieve functionality, mutations would next be made in the regions involved in G protein binding. Mutations would be made in regions involved in ligand binding only as a last resort, and then an effort would be made to preserve ligand binding by making conservative substitutions whenever possible.

Preferably, the yeast genome is modified so that it is unable to produce the yeast receptors which are homologous to the exogenous receptors in functional form. Otherwise, a positive assay score might reflect the ability of a peptide to activate the endogenous G protein-coupled receptor, and not the receptor of interest.

(i). Chemoattractant Receptors

The N-formyl peptide receptor is a classic example of a calcium mobilizing G protein-coupled receptor expressed by neutrophils and other phagocytic cells of the mammalian immune system (Snyderman et al. (1988) In Inflammation: Basic Principles and Clinical Correlates, pp. 309-323). N-Formyl peptides of bacterial origin bind to the receptor and engage a complex activation program that results in directed cell movement, release of inflammatory granule contents, and activation of a latent NADPH oxidase which is important for the production of metabolites of molecular oxygen. This pathway initiated by receptor-ligand interaction is critical in host protection from pyogenic infections. Similar signal transduction occurs in response to the inflammatory peptides C5a and IL-8.

Two other formyl peptide receptor like (FPRL) genes have been cloned based on their ability to hybridize to a fragment of the NFPR cDNA coding sequence. These have been named FPRL1 (Murphy et al. (1992) J. Biol Chem. 267:7637-7643) and FPRL2 (Ye et al. (1992) Biochem Biophys Res. Comm. 184:582-589). FPRL2 was found to mediate calcium mobilization in mouse fibroblasts transfected with the gene and exposed to formyl peptide. In contrast, although FPRL1 was found to be 69% identical in amino acid sequence to NFPR, it did not bind prototype N-formyl peptides ligands when expressed in heterologous cell types. This lead to the hypothesis of the existence of an as yet unidentified ligand for the FPRL1 orphan receptor (Murphy et al. supra).

(ii.) G Proteins

In the case of an exogenous Gprotein-coupled receptor, the yeast cell must be able to produce a G protein which is activated by the exogenous receptor, and which can in turn activate the yeast effector(s). The art suggests that the endogenous yeast Gα subunit (e.g., GPA) will be often be sufficiently homologous to the “cognate” Gα subunit which is natively associated with the exogenous receptor for coupling to occur. More likely, it will be necessary to genetically engineer the yeast cell to produce a foreign Gα subunit which can properly interact with the exogenous receptor. For example, the Gα subunit of the yeast G protein may be replaced by the Gα subunit natively associated with the exogenous receptor.

Dietzel and Kuian, (1987) Cell, 50:1001) demonstrated that rat Gαs functionally coupled to the yeast Gβγ complex. However, rat Gαi2 complemented only when substantially overexpressed, while Gα0 did not complement at all. Kang, et al., Mol. Cell. Biol., (1990)10:2582). Consequently, with some foreign Gα subunits, it is not feasible to simply replace the yeast Gα.

If the exogenous G protein coupled receptor is not adequately coupled to yeast Gβγ by the Gα subunit natively associated with the receptor, the Gα subunit may be modified to improve coupling. These modifications often will take the form of mutations which increase the resemblance of the Gα subunit to the yeast Gα while decreasing its resemblance to the receptor-associated Gα. For example, a residue may be changed so as to become identical to the corresponding yeast Gα residue, or to at least belong to the same exchange group of that residue. After modification, the modified Gα subunit might or might not be “substantially homologous” to the foreign and/or the yeast Gα subunit.

The modifications are preferably concentrated in regions of the Gα which are likely to be involved in Gβγ binding. In some examples, the modifications will take the form of replacing one or more segments of the receptor-associated Gα with the corresponding yeast Gα segment(s), thereby forming a chimeric Gα subunit. (For the purpose of the appended claims, the term “segment” refers to three or more consecutive amino acids.) In other examples, point mutations may be sufficient.

This chimeric Gα subunit will interact with the exogenous receptor and the yeast Gβγ complex, thereby permitting signal transduction. While use of the endogenous yeast Gβγ is preferred, if a foreign or chimeric Gβγ is capable of transducing the signal to the yeast effector, it may be used instead.

Although many of the techniques presented above require specific knowledge of a receptor active in a particular biological pathway, it will be recognized by those skilled in the art that such knowledge is not required for the screening of a library of chimeric polypeptides of the present invention. Rather, cell-based assays are well known in the art in which cells of a selected phenotype can be used to screen chimeric polypeptides for those which induce a particular alteration in the phenotype. In this way, chimeric polypeptides can be found that have a desired biological function that is not understood on a molecular level.

Exemplification

The invention now being generally described, it will be more readily understood by reference to the following examples which are included merely for purposes of illustration of certain aspects and embodiments of the present invention, and are not intended to limit the invention.

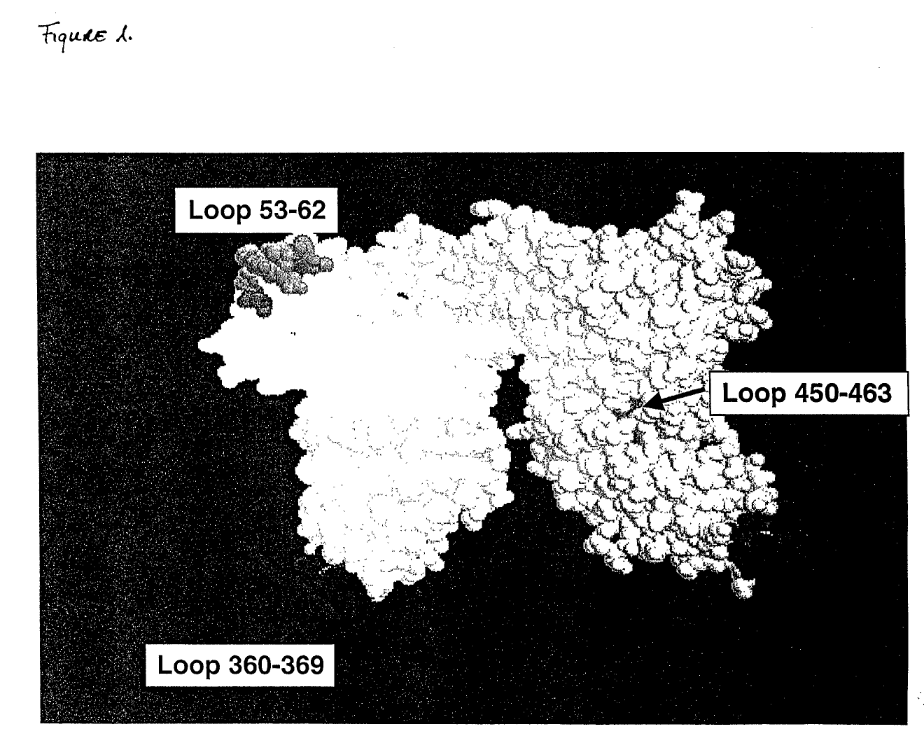

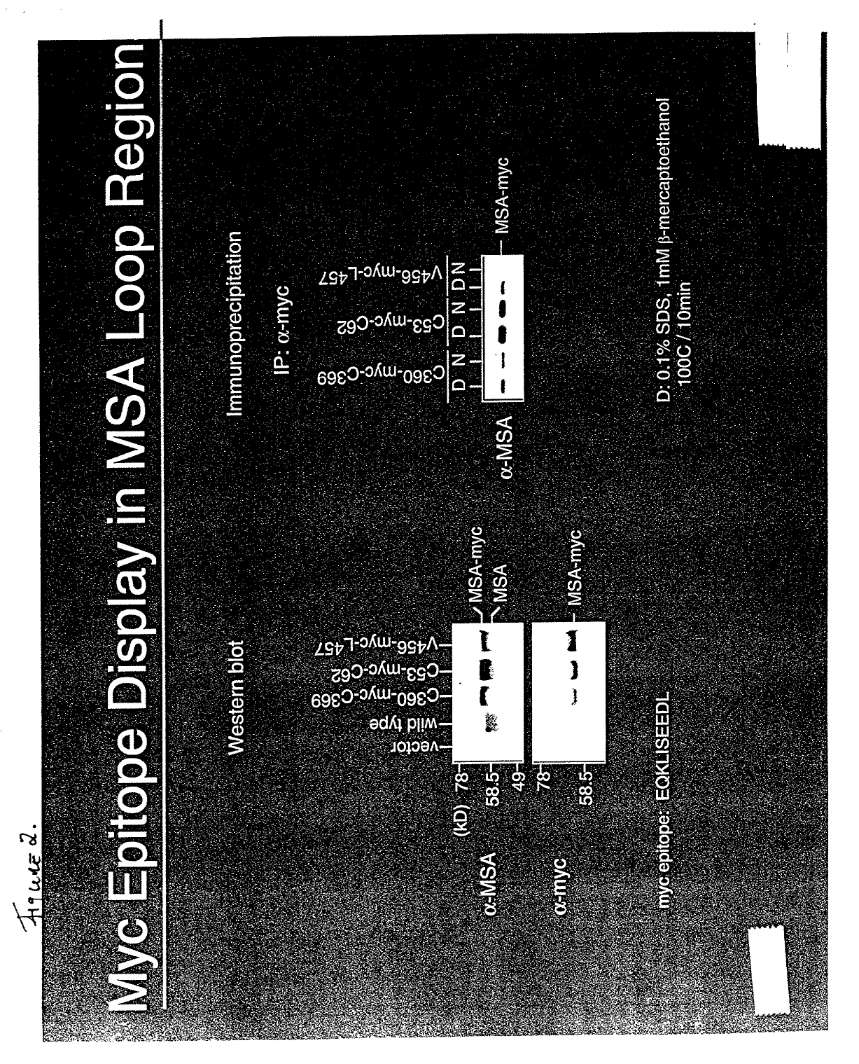

Serum albumin loop regions. A space-filling model of human serum albumin (HSA) is shown in FIG. 1. The tertiary structure of HSA reveals the presence of ten approximate helical regions or loops, each constrained by disulfide bonded cysteine pairs. The space-filling model was used to predict loop regions that are exposed on the surface of the protein. Two amino acid segments were chosen to represent surface exposed regions (loop 53-62 and loop 360-369) and a third to represent a region assumed to be buried within the protein (loop 450-463). These and other candidate loops (Cys⁵³-Cys⁶², Cys⁷⁵-Cys⁹¹, Cys⁹⁰-Cys¹⁰¹, Cys²⁴⁵-Cys²⁵³, Cys²⁶⁶-Cys²⁷⁹, Cys³⁶⁰-Cys³⁶⁹, Cys⁴⁶¹-Cys⁴⁷⁷, Cys⁴⁷⁶-Cys⁴⁸⁷, and Cys⁵⁵⁸-Cys⁵⁶⁷) are depicted in FIGS. 4A-I.

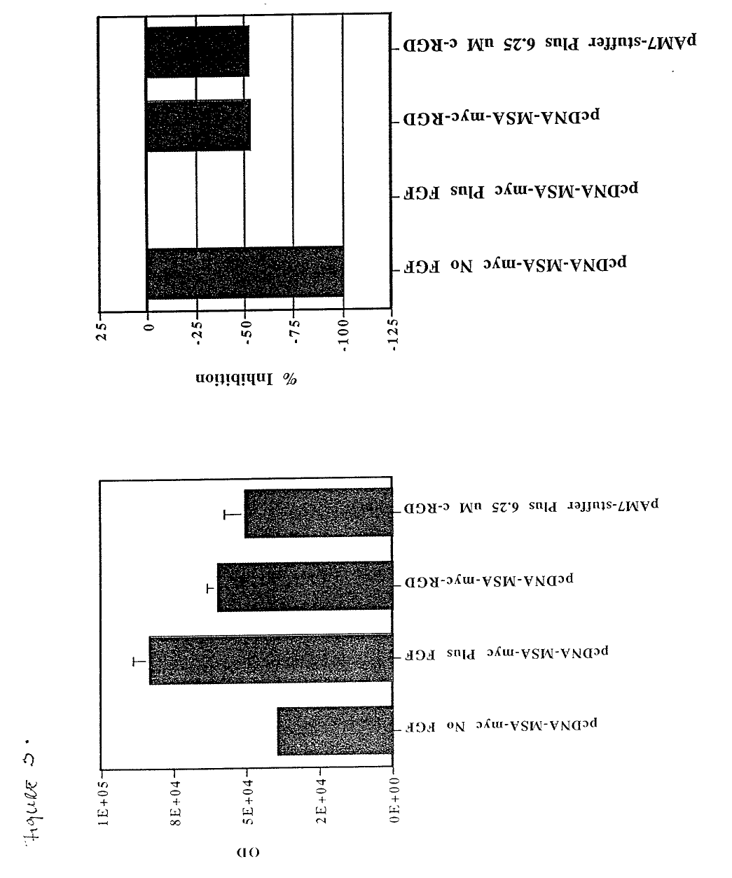

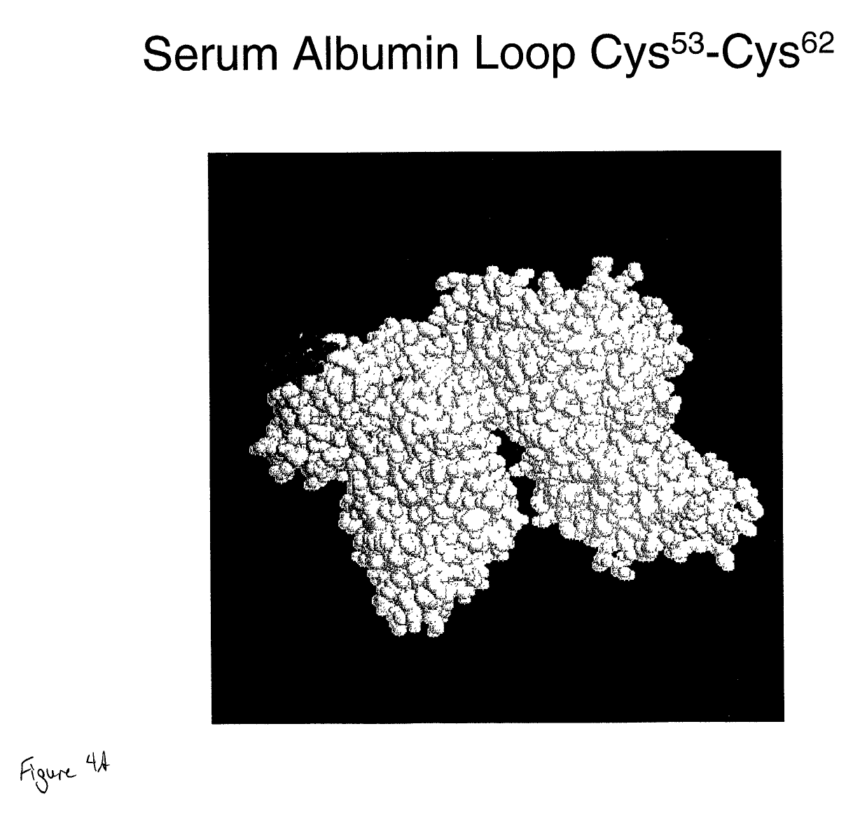

Myc epitope display in MSA loop regions. In order to determine whether the predicted loops were indeed exposed on the surface of the albumin molecule, mouse serum albumin (MSA) was modified to include the myc epitope, EQKLISEEDL. The myc epitope was inserted in the middle of each of three amino acid segments: between amino acids 57-58 for loop 53-62, amino acids 364-365 for loop 360-369 and amino acids 467-468 for loop 450-467. Cos7 cells were transfected with either wild type MSA or the various myc containing MSA constructs. The presence of the proteins in the medium was first determined by Western blot analysis using antibodies specific for MSA and the myc epitope. As can be seen in the left half of FIG. 2, only samples from media from cells transfected with MSA or MSA-Myc reveal the presence of the albumin protein. Additionally, only the samples from cells transfected with MSA-Myc are positive for the myc epitope. As the samples are all denatured by virtue of the SDS-PAGE system, this analysis does not allow for the differentiation of myc epitopes that would be exposed on the surface versus one that was buried within the protein. For this analysis immunoprecipitation with the myc-specific antibody was utilized. In this experiment, the conditioned media was either mixed directly with the antibody (N, native) or first denatured in the presence of 0.1% SDS, 1 mM β-mercapthoethanol and heat (100° C. for 10 min) and then antibody added (D, denatured). Following immunoprecipitation the presence of the proteins that could be precipitated by the myc antibody were revealed by Western blot analysis using the MSA specific antibody. The right panel of FIG. 2 shows that, as predicted, the albumin proteins with myc inserted in loops 53-62 and 360-369 were bound by the myc antibody regardless of whether the protein was in its native or denatured form. On the other hand, when myc was inserted in the predicted buried region, loop 450-463, the protein only bound the antibody when it was first denatured. This experiment clearly demonstrates that loops 53-62 and 360-369 are exposed on the surface of the MSA protein and therefore good for display. Additionally, the 450-463 loop is buried.

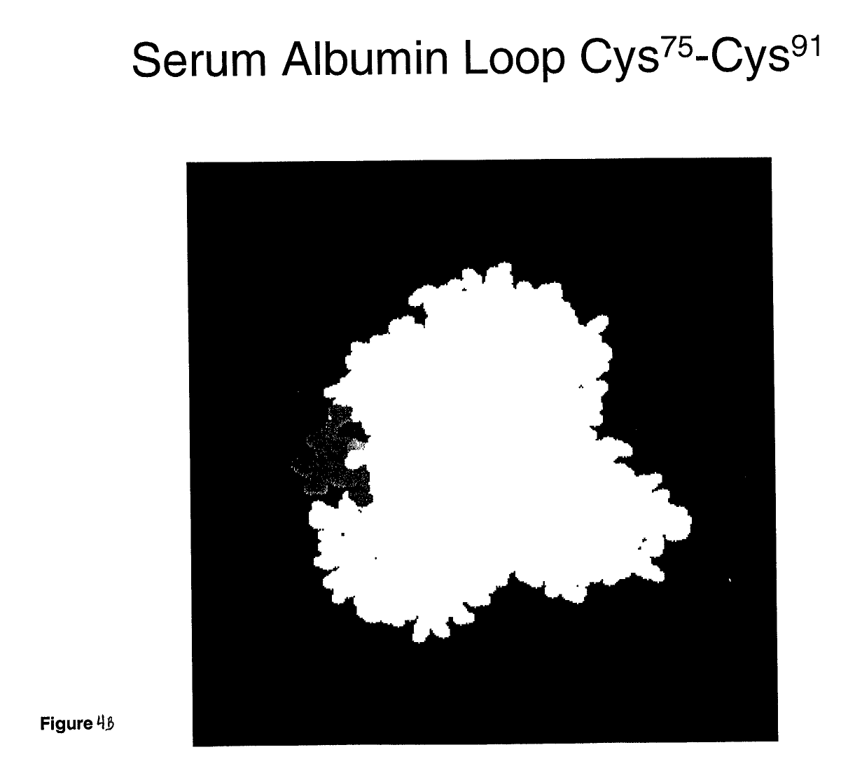

Inhibition of bovine capillary endothelial cells (BCE) MSA-RGD. The goal of this experiment was determine the function of MSA with the RGD peptide (VRGDF) displayed on the surface of the protein in the loop 53-58 region (MSA-myc-RGD). RGD was chosen, as this peptide can efficiently bind to αvβ3 integrin receptors on endothelial cells and inhibit their proliferation. Triplicate wells of Cos7 cells were transfected with the following constructs: MSA-myc (the myc epitope was added to the C-terminal tail of MSA in this iteration); MSA-myc-RGD; or pAM7-stuffer. These Cos7 cells were grown in the lower chamber of a Transwell® tissue culture plate with BCE cells in the upper chamber. To stimulate growth of the BCE cells, FGF was added to the lower chamber or not in the case of no FGF control and the cells allowed to grow for 72 hours. To one set of wells, those with pAM7-stuffer, 6.25 μM c-RGD peptide was also added. Cell growth was determined by a Calcein-binding fluorescence assay. The left panel of FIG. 3 is a graph of the optical density (OD) for each. The data reveals the addition of FGF results in a 2-fold stimulation of growth of the BCE cells. This growth was inhibited by the c-RGD peptide and also by the secreted MSA-myc-RGD protein. The right panel is a different way of looking at the same data. In this instance the degree of inhibition of growth is graphed for each. The data shows that the MSA-Myc-RGD protein inhibited the growth of the BCE cell by 53% and the degree of inhibition was equivalent to that of the added RGD peptide. The RGD peptide displayed on the surface of the MSA molecule inhibited BCE cell growth as efficiently as the endogenously added free RGD peptide demonstrating that the peptide retains its activity in the looped orientation.

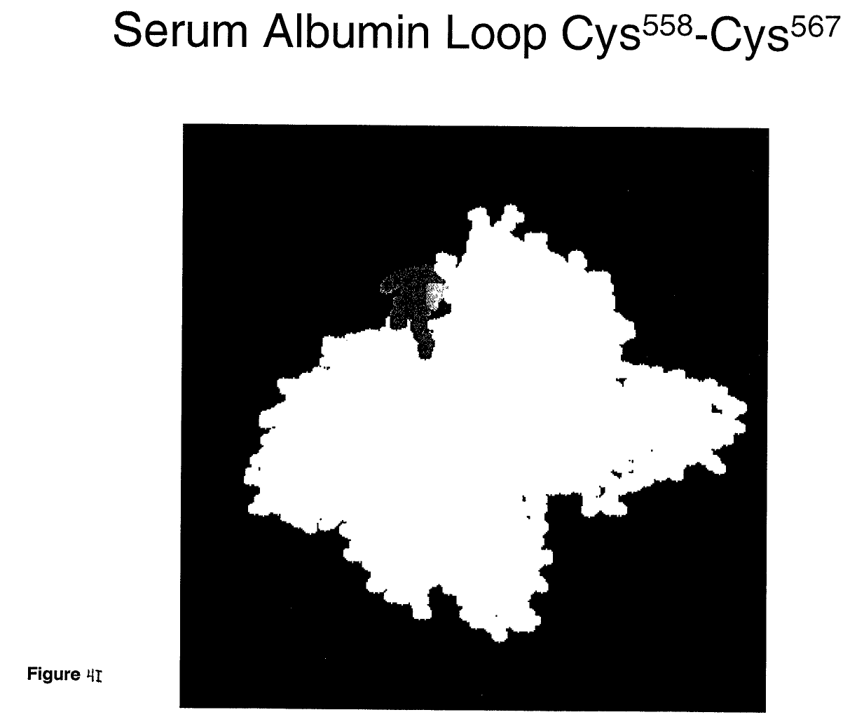

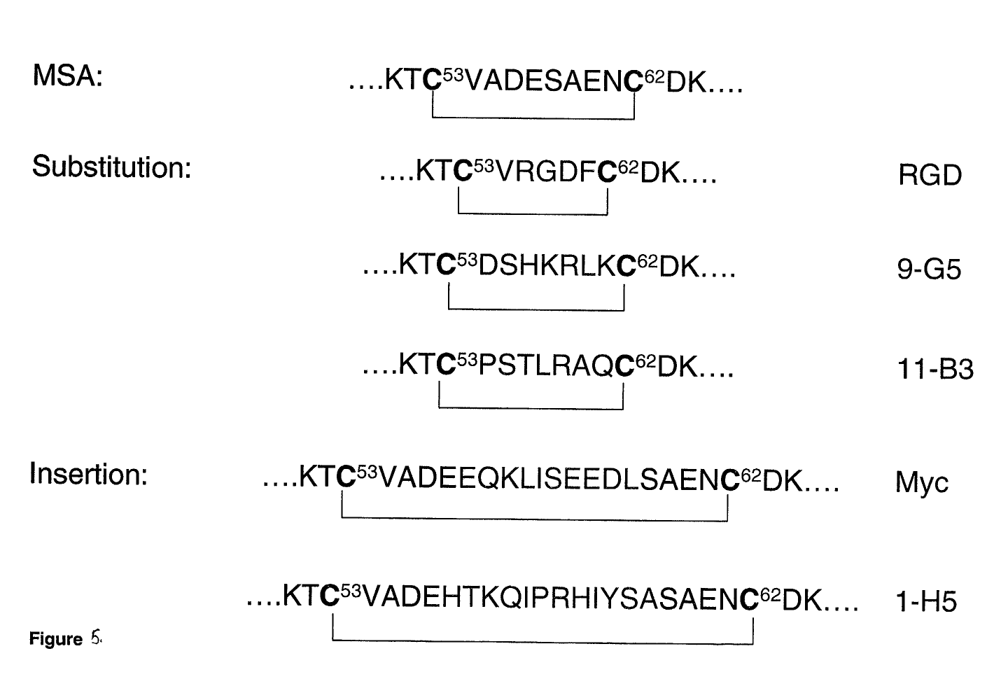

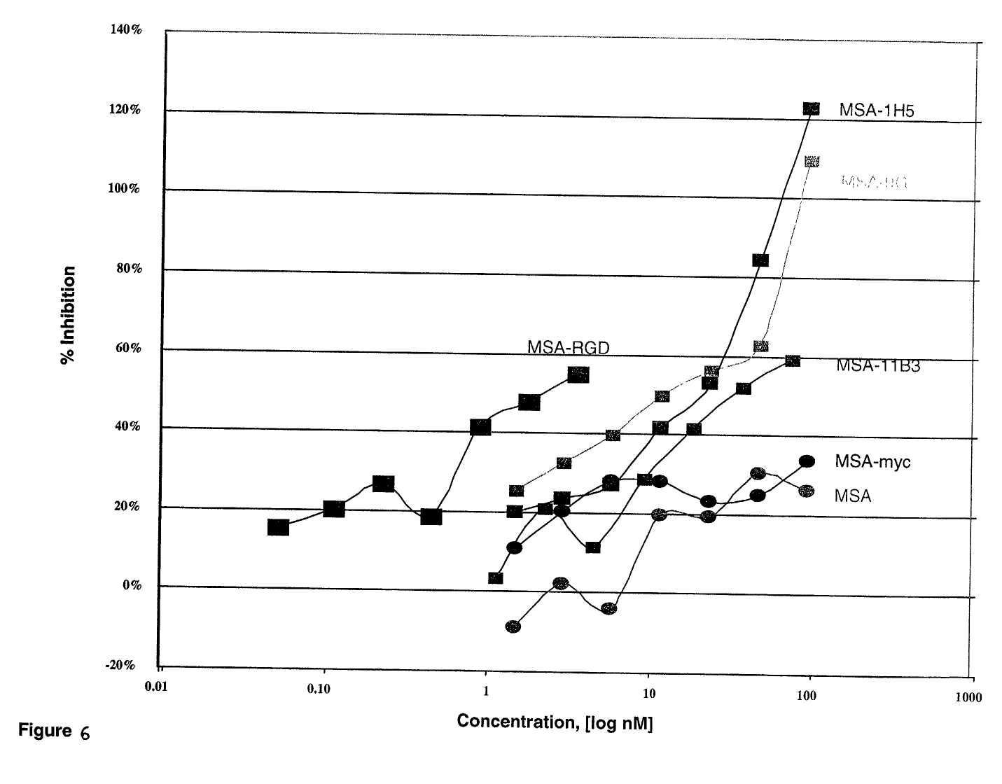

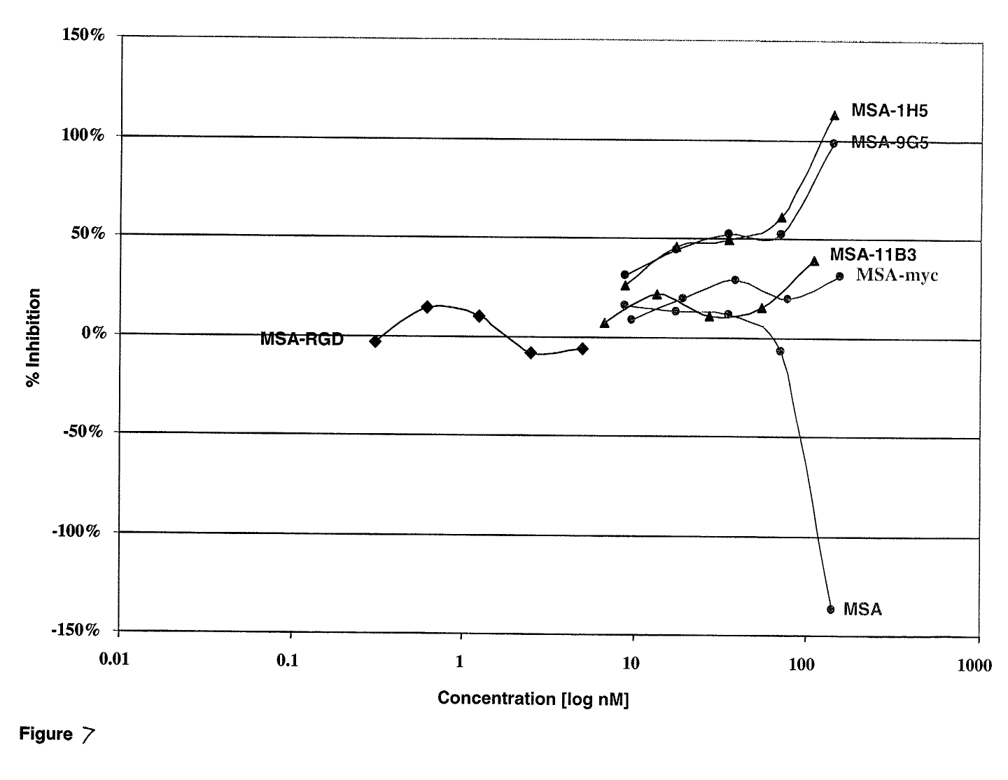

Inhibition of BCE and HUVEC proliferation by serum albumin-EC binding peptide fusions. This experiment was designed to demonstrate the inhibition of BCE and HUVEC cell proliferation by purified mouse serum albumin (SA) proteins that displayed endothelial cell binding (EC) peptides. In the MSA-peptide fusions the peptide sequence was inserted into a cysteine constrained loop between amino acids 53 and 62. The proteins were produced by COS-7 cells that were transfected with expression plasmids that directed the synthesis and secretion of the particular recombinant protein. As shown in FIG. 5, in the MSA-9G5, MSA-11B3 and MSA-RGD constructs the inserted peptides replaced the naturally occurring residues of MSA between cys53-cys62. In MSA-1H5 and MSA-myc constructs (negative control), the peptides were inserted into the loop at amino acid glu57. FIGS. 6 and 7 show the inhibitory effect of the purified proteins on the proliferation of BCE and HUVEC cells that were stimulated by FGF.

Experimental Design of the EC Proliferation Experiments

Protein production and concentration

COS7-L cells were transfected with protein expression constructs expressing:

1. MSA, full-length mouse serum albumin (negative control)

2. MSA-RGD, in which the RGD sequence (VRGDF) replaces the MSA sequence between Cys 53 and Cys 62

3. MSA-11B3, in which the 11-B3 peptide sequence (PSTLRAQ) replaces the MSA sequence between Cys 53 and Cys 62

4. MSA-1H5, in which the 1-H5 peptide sequence (HTKQIPRHIYSA) is inserted between Glu 57 and Ser 58 within the Cys 53 and Cys 62 loop of MSA

5. MSA-9G5, in which the 9-G5 peptide sequence (DSHKRLK) replaces the MSA sequence between Cys 53 and Cys 62

6. MSA-myc, in which the Myc epitope peptide sequence (EQKLISEEDL) is inserted between Glu 57 and Ser 58 within the Cys 53 and Cys 62 loop of MSA (negative control)

The transfected COS7-L cells were cultured in defined serum-free media (VP-SFM). Each day for 5 days, the conditioned media were collected from the cells, centrifuged to remove dead cells and other cellular debris, and then frozen. The 5 days-worth of cultured media were pooled and concentrated 500-fold using a Centiprep-80 with a molecular weight cut-off of 50 (for MSA, MSA-RGD, MSA-9G5) or a molecular weight cut-off of 30 (for MSA-myc, MSA-11B3, MSA-1H5). The concentration of the albumin proteins was determined by Western blot analysis of each preparation using a rabbit anti-MSA antibody and using purified MSA of known concentration to generate a standard curve. Following development of the blot and exposure to film the autoradiographs were analyzed using the Gel Doc 1000 image analysis system and Molecular Analyst software (BioRad).

BCE Proliferation Assays

On day zero, bovine capillary endothelial cells (BCE) at passage 11 were plated in 96-well tissue culture plates at a density of 2×103 cells per well in 100 ml 5% calf serum (CS)/DMEM supplemented with penicillin/streptomycin (PS). The cells were then incubated overnight in an atmosphere of 10% CO₂, 37° C.

On day one, the media was changed to 150 ml 2% CS/DMEM/PS. The albumin proteins were added to the first well as 8.75 ml which contains an additional 150 ml of 2% CS/DMEM/PS. 150 ml was then removed from this well and added to the next well resulting in a 1:2 dilution of the protein. This process was repeated for a total of six times each in triplicate. 50 ml of 4 ng/ml FGF (final concentration: 1 ng/ml FGF) was then added to each well and the plates incubated as above for 72 h. A synthetic peptide of cyclic RGD (c-RGD) at a concentration of 4.1 mM was included to serve as a positive control for inhibition of proliferation. Cells without addition of protein but with FGF added and without FGF added were included on each plate as additional controls.

After the 72 h incubation, the media was removed, and the plates were washed twice with PBS and frozen at −80° C. Proliferation of the BCE cells was assessed using the CyQUANT® cell proliferation assay kit according to the manufacturer's recommendations.

Conclusions

The insertion of the EC binding peptides into MSA increased their inhibitory activity by approximately 1000-fold. The MSA-EC binding peptide fusions inhibited BCE and HUVEC proliferation in the nanomolar (nM) range while the synthetic peptides were active in the micromolar (mM) range. The control MSA and MSA-myc proteins did not significantly affect the proliferation of the target endothelial cells.

Induction of tumor cell apoptosis by MSA-RGD fusions. Peptides containing the RGD (Arg-Gly-Asp) motif have been shown to induce apoptosis in a caspase-3 dependent manner through the promotion of pro-caspase3 auto-cleavage and activation (Buckley et al., 1999). It was therefore of interest to determine if the MSA-RGD fusion was also capable of inducing apoptosis. To test this hypothesis, human non-small cell lung carcinoma cells (NCI 1869) were plated on the membrane of a transwell insert. These cells were incubated to allow attachment. Cos7 cells were transfected with a plasmid containing cDNA encoding (pcDNA MSA-RGD/53) for the expression and secretion of the respective fusion protein. An empty vector (pcDNA3) was transfected in parallel as a negative control. After 24 hours, the transwell insert carrying the NCI-1869 cells was transferred to the plate containing the Cos7/MSA-RGD transfectants. The cells were co-incubated for an additional 24 hours. The NCI-1869 cells were then recovered and incubated in PBS/Mg++ containing the fluorometric Caspase-3 substrate, DEVD-AFC. Cleavage of this fluorogenic tetrapeptide substrate by Caspase-3 generates a fluorescent signal, which is read in a fluorometric plate reader as a measure of the induction of apoptosis.

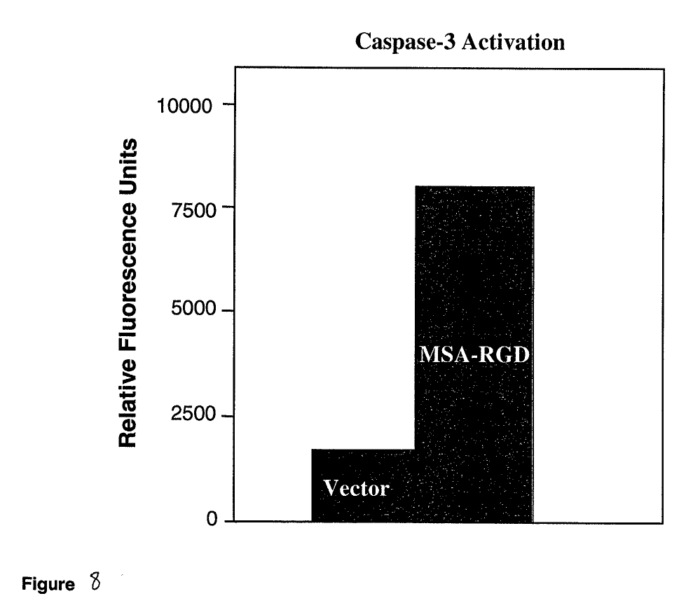

The results presented in FIG. 8 (each bar is the average of 3 independent samples) demonstrate that the secretion of MSA-RGD by Cos7 cells leads to a 4.9 fold induction of apoptosis relative to the vector control in NCI-1869 cells. Incubation of these cells with purified RGD peptide also leads to the induction of apoptosis as assessed by microscopic analysis.

The skilled artisan will recognize many equivalents to the disclosed invention, all of which are intended to be within the scope of the present invention. All articles, patents, and applications cited above are incorporated herein by reference.

We claim:

1. A chimeric polypeptide comprising a serum albumin protein (SA) having a biologically active heterologous peptide sequence inserted therein.

2. A chimeric polypeptide having the structure A-B-C, wherein:
A represents a first fragment of serum albumin (SA);
B represents a biologically active heterologous peptide sequence; and
C represents a second peptide fragment of SA.

3. A chimeric polypeptide comprising:
a first peptide fragment, comprising an N-terminal fragment of serum albumin (SA) protein;
a second peptide fragment, comprising a biologically active heterologous peptide sequence, and
a third peptide fragment, comprising a C-terminal fragment of SA.

4. The chimeric polypeptide of claim 1, 2, or 3, wherein the heterologous peptide sequence comprises a fragment of an angiogenesis-inhibiting protein or polypeptide.

5. The chimeric polypeptide of claim 4, wherein said angiogenesis-inhibiting protein or polypeptide is selected from the group consisting of angiostatin, endostatin, and peptide fragments thereof.

6. The chimeric polypeptide of claim 1, 2, or 3, wherein the heterologous peptide sequence binds to a cell surface receptor protein.

7. The chimeric polypeptide of claim 6, wherein the receptor protein is a G-protein coupled receptor.

8. The chimeric polypeptide of claim 6, wherein the receptor protein is a tyrosine kinase receptor.

9. The chimeric polypeptide of claim 6, wherein the receptor protein is a cytokine receptor.

10. The chimeric polypeptide of claim 6, wherein the receptor protein is an MIRR receptor.

11. The chimeric polypeptide of claim 6, wherein the receptor protein is an orphan receptor.

12. The chimeric polypeptide of claim 1, 2, or 3, wherein the chimeric polypeptide binds to an extracellular receptor or an ion channel.

13. The chimeric polypeptide of claim 12, wherein the chimeric polypeptide is an agonist of said receptor or ion channel.

14. The chimeric polypeptide of claim 12, wherein the chimeric polypeptide is an antagonist of said receptor or ion channel.

15. The chimeric polypeptide of claim 1, 2, or 3, wherein the chimeric polypeptide induces apoptosis.

16. The chimeric polypeptide of claim 1, 2, or 3, wherein the chimeric polypeptide modulates cell proliferation.

17. The chimeric polypeptide of claim 1, 2, or 3, wherein the chimeric polypeptide modulates differentiation of cell types.

18. The chimeric polypeptide of claim 1, 2, or 3, wherein the heterologous peptide sequence comprises between 4 and 400 residues.

19. The chimeric polypeptide of claim 1, 2, or 3, wherein the heterologous peptide sequence comprises between 4 and 200 residues.

20. The chimeric polypeptide of claim 1, 2, or 3, wherein the heterologous peptide sequence comprises between 4 and 100 residues.

21. The chimeric polypeptide of claim 1, 2, or 3, wherein the heterologous peptide sequence comprises between 4 and 20 residues.

22. The chimeric polypeptide of claim 1, 2, or 3, wherein the tertiary structure of the chimeric polypeptide is similar to the tertiary structure of native SA.

23. The chimeric polypeptide of claim 1, wherein the inserted peptide sequence replaces a portion of native SA sequence.

24. The chimeric polypeptide of claim 23, wherein the inserted peptide sequence and the replaced portion of native SA sequence are of unequal length.

25. The chimeric polypeptide of claim 1, 2, or 3, wherein the half-life of the polypeptide in the blood is no less than 14 days.

26. The chimeric polypeptide of claim 1, 2, or 3, wherein the half-life of the polypeptide in the blood is no less than 10 days.

27. The chimeric polypeptide of claim 1, 2, or 3, wherein in the half-life of the polypeptide in the blood is no less than 50% of the half-life of native SA.

28. A nucleic acid encoding the chimeric polypeptide of claim 1, 2, or 3.

29. A delivery vector comprising the nucleic acid of claim 28.

30. The delivery vector of claim 29, wherein said delivery vector comprises a virus or retrovirus.

31. The delivery vector of claim 30, wherein said virus or retrovirus is selected from the group consisting of adenoviruses, adeno-associated viruses, herpes simplex viruses, human immunodeficiency viruses, or vaccinia viruses.

32. Transfected cells comprising target cells which have been exposed to the delivery vector of claim 29.

33. The transfected cells of claim 32, wherein the cells are selected from the group consisting of blood cells, skeletal muscle cells, stem cells, skin cells, liver cells, secretory gland cells, hematopoietic cells, and marrow cells.

34. A pharmaceutical preparation comprising a pharmaceutically acceptable excipient and the chimeric polypeptide of claim 1, 2, or 3.

35. A method for treating disease in an organism, comprising administering as a pharmaceutical preparation to the organism the chimeric polypeptide of claim 1, 2, or 3.

36. A method for treating disease in an organism, said method comprising:
providing a delivery vector comprising genetic material which encodes the chimeric polypeptide of claim 1, 2, or 3; and
introducing said vector into target cells in vivo, under conditions sufficient to induce said target cells to express said polypeptide.

37. A method for treating a disease in an organism comprising:
providing a delivery vector comprising genetic material which encodes the chimeric polypeptide of claim 1, 2, or 3;
introducing said vector into target cells ex vivo; and
introducing said target cells containing the introduced vector into the organism under conditions sufficient to induce said target cells to express said polypeptide.

38. The method of claim 36 or 37, wherein the target cells are selected from the group consisting of blood cells, skeletal muscle cells, stem cells, skin cells, liver cells, secretory gland cells, hematopoietic cells, and marrow cells.

39. A chimeric polypeptide having the structure (A-B-C)ₙ, wherein:
A, independently for each occurrence, represents a fragment of serum albumin (SA);
B, independently for each occurrence, represents a biologically active heterologous peptide sequence;
C, independently for each occurrence, represents a second biologically active heterologous peptide sequence or a fragment of serum albumin (SA); and
n is an integer greater than 0.

40. The polypeptide of claim 39, wherein B and C comprise identical sequences.

41. The polypeptide of claim 39, wherein B and C comprise fragments of a single protein.

42. The polypeptide of claim 39, wherein B and C comprise fragments two different proteins.

43. A chimeric polypeptide comprising serum albumin protein (SA) having at least two biologically active heterologous peptide sequences inserted therein.

44. The polypeptide of claim 43, wherein the heterologous peptide sequences are identical.

45. The polypeptide of claim 43, wherein the heterologous peptide sequences comprise distinct sequences of a protein.

46. The polypeptide of claim 44, wherein the heterologous peptide sequences comprise sequences from at least two different proteins.

47. A method for modulating one or more of cell proliferation, cell differentiation, and cell death in an organism, comprising administering as a pharmaceutical preparation to the organism the chimeric polypeptide of claim 1, 2, or 3.

48. A method for modulating one or more of cell proliferation, cell differentiation, and cell death in an organism, comprising:
providing a delivery vector comprising genetic material which encodes the chimeric polypeptide of claim 1, 2, or 3; and
introducing said vector into target cells in vivo, under conditions sufficient to induce said target cells to express said polypeptide.

49. The chimeric polypeptide of claim 1, wherein the biologically active heterologous peptide sequence is inserted into a cysteine loop of the serum albumen protein.

50. The chimeric polypeptide of claim 49, wherein the cysteine loop is selected from Cys⁵³-Cys⁶², Cys⁷⁵-Cys⁹¹, Cys⁹⁰-Cys¹⁰¹, Cys²⁴⁵-Cys²⁵³, Cys²⁶⁶-Cys²⁷⁹, Cys³⁶⁰-Cys³⁶⁹, Cys⁴⁶¹-Cys⁴⁷⁷, Cys⁴⁷⁶-Cys⁴⁸⁷, and Cys⁵⁵⁸-Cys⁵⁶⁷.

51. The chimeric polypeptide of claim 23, wherein the biologically active heterologous peptide sequence replaces a portion of a cysteine loop of the serum albumen protein.

52. The chimeric polypeptide of claim 51, wherein the cysteine loop is selected from Cys⁵³-Cys⁶², Cys⁷⁵-Cys⁹¹, Cys⁹⁰-Cys¹⁰¹, Cys²⁴⁵-Cys²⁵³, Cys²⁶⁶-Cys²⁷⁹, Cys³⁶⁰-Cys³⁶⁹, Cys⁴⁶¹-Cys⁴⁷⁷, Cys⁴⁷⁶-Cys⁴⁸⁷, and Cys⁵⁵⁸-Cys⁵⁶⁷.

53. The chimeric polypeptide of claim 50 or 52, wherein the cysteine loop is selected from Cys⁵³-Cys⁶², Cys⁷⁵-Cys⁹¹, Cys⁹⁰-Cys¹⁰¹, Cys²⁴⁵-Cys²⁵³, Cys²⁶⁶-Cys²⁷⁹, Cys³⁶⁰-Cys³⁶⁹, Cys⁴⁶¹-Cys⁴⁷⁷, Cys⁴⁷⁶-Cys⁴⁸⁷, and Cys⁵⁵⁸-Cys⁵⁶⁷.
